SEQUENCE LISTING (1 sequence)

SEQ ID NO: 1 - protein, 147 aa, Synthetic construct
  DISULFID 97..147: note = Disulfide bridge: 97..147
MVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

(19) United States
(12) Patent Application Publication
(10) Pub. No.: US 2026/0007721 A1
(43) Pub. Date: Jan. 8, 2026

(54) LEPTIN THERAPY FOR GLUCOSE METABOLISM DISORDERS

(71) Applicant: Dignity Health, San Francisco, CA (US)

(72) Inventor: Zaman Mirzadeh, San Francisco, CA (US)

(21) Appl. No.: 19/263,177

(22) Filed: Jul. 8, 2025

Related U.S. Application Data
(60) Provisional application No. 63/668,657, filed on Jul. 8, 2024.

(51) Int. Cl. A61K 38/22; A61K 47/64; A61P 3/10; C07K 16/28

(52) U.S. Cl. CPC A61K 38/2264; A61K 47/64; A61P 3/10; C07K 16/2869

(57) ABSTRACT

The present disclosure provides methods for treating, ameliorating, and/or preventing glucose metabolism disorder by administering a leptin active delivered to the central nervous system (CNS), and in particular to brain regions. Also provided are compositions, kits and systems for practicing the methods.

### CROSS-REFERENCE TO RELATED APPLICATIONS

This application claims priority from U.S. Provisional Patent Application No. 63/668,657, filed on Jul. 8, 2024, the entire contents of which are incorporated by reference.

### SUBMISSION OF A SEQUENCE LISTING ON ASCII TEXT FILE

The present application contains a Sequence Listing which has been submitted electronically in .XML format and is hereby incorporated herein by reference in its entirety. Said computer readable file was created on Jul. 5, 2025, named 102695-851179 Sequence Listing.xml and is 4,096 bytes in size.

### FIELD OF THE INVENTION

The present disclosure relates generally to medical treatment of glucose metabolism disorders, such as diabetes or hyperglycemia, including Type 1 diabetes.

### BACKGROUND OF THE INVENTION

Type 1 Diabetes (T1D) is among the most common chronic diseases of childhood, afflicting over 1.5 million Americans including 250,000 children and adolescents, and is associated with over $16 billion dollars per year in healthcare costs. T1D incidence in the U.S. has risen steadily over the last few decades with the current disease growth rate at four times the population growth rate. Exacerbating the problem, fewer than a third of people with T1D consistently achieve target blood glucose control levels, with the remainder at risk for secondary health complications. These mounting concerns emphasize the need for novel treatment strategies as an alternative to insulin therapy, the current mainstay treatment for T1D. Current therapies for diabetes require very active participation by the patient to monitor their blood glucose level and administer insulin in a reactive manner, with insulin dosing adapted not only to the blood glucose level, but also the anticipated nutritional state and activity level. An urgent need exists for new and improved T1D treatments.

### SUMMARY OF THE INVENTION

One aspect of the present disclosure encompasses a method of treating a glucose metabolism disorder in a subject in need thereof, the method comprising administering to the subject a therapeutically effective amount of a leptin active, wherein the administering comprises delivering the leptin active to the central nervous system. In one aspect, the glucose metabolism disorder is selected from hyperglycemia, Type I diabetes, Type II diabetes, and any combination thereof.

In any of the methods, the leptin active is selected from the group consisting of a leptin compound, a modified leptin compound, a pharmaceutically acceptable salt thereof, a crystal form thereof, an enantiomer thereof, and any combination thereof. The leptin compound is selected from a leptin, a leptin isomer, a leptin derivative, a leptin analog, a leptin mimetic, a leptin agonist, an anti-leptin receptor antibody, or any combination thereof, and the modified leptin compound is a conjugate between the leptin compound and a Blood-Brain-Barrier (BBB) penetrating moiety. In one aspect, the BBB penetrating moiety comprises at least one fragment selected from the group consisting of a transferrin receptor, an insulin receptor, an insulin-like growth factor receptor, a low density lipoprotein receptor, a low density lipoprotein receptor-related protein 8, a low density lipoprotein receptor-related protein 1, a heparin-binding epidermal growth factor-like growth factor, an antibody thereof, or any combination thereof.

In any of the methods, delivery to the central nervous system comprises delivery to a region of the brain or the spinal cord. In one aspect, the region of the brain includes but is not limited to the arcuate nucleus, the ventromedial nucleus of the hypothalamus, or the nucleus of the solitary tract of the hindbrain. In one aspect, the therapeutically effective amount of the leptin active is about 5.0 mcg/kg/day to about 10.0 mcg/kg/day. In various aspects, the delivery is once daily, once every two days, once every three days, once every four days, once every five days, once every six days, weekly or monthly. In yet another aspect, the delivery is selected from once daily over a period of time in a fixed dose, once daily over a period of time in ascending doses, once daily over a period of time in descending doses, and once daily over a period of time in fluctuating doses. In one aspect, delivery to the delivery to the central nervous system is achieved by systemic delivery of the leptin active to the subject. In one aspect, the systemic delivery is achieved by a route of administration selected from intravenous, intramuscular, subcutaneous, oral, buccal and transdermal. In another aspect, the delivery is to a region of the spinal cord, including but not limited to the lumbar region or the cervical region. In yet another aspect, the delivery is achieved via a catheter, a reservoir, or a pump. In one aspect, the delivery is through an intrathecal catheter. In yet another aspect, the administering continues for a period of at least 5 days, at least 6 days, least 7 days, at least 8 days, least 9 days, at least 10 days, at least 11 days, or at least 12 days, or longer.

Another aspect of the present disclosure encompasses a pharmaceutical composition comprising: (i) a leptin active; and (ii) at least one excipient capable of effectuating and/or enhancing the brain uptake of the leptin active. In one aspect, the leptin active is selected from the group consisting of a leptin compound, a modified leptin compound, a pharmaceutically acceptable salt thereof, a crystal form thereof, an enantiomer thereof, and any combination thereof. The leptin compound is selected from a leptin, a leptin isomer, a leptin derivative, a leptin analog, a leptin mimetic, a leptin agonist, an anti-leptin receptor antibody, or any combination thereof, and the modified leptin compound is a conjugate between the leptin compound and a Blood-Brain-Barrier (BBB) penetrating moiety. In one aspect, the BBB penetrating moiety comprises at least one fragment selected from the group consisting of a transferrin receptor, an insulin receptor, an insulin-like growth factor receptor, a low density lipoprotein receptor, a low density lipoprotein receptor-related protein 8, a low density lipoprotein receptor-related protein 1, a heparin-binding epidermal growth factor-like growth factor, an antibody thereof, or any combination thereof. In another aspect, the at least one excipient is selected from the group consisting of an osmotic agent, an osmogenic agent, a sugar, an inorganic salt, and any combination thereof. In yet another aspect, the pharmaceutical composition comprises at least one excipient comprising mannitol, glycerol, lactulose, sorbitol, polyethylene glycol, glutamic acid, glycine, polysorbate 20, sucrose, sodium chloride, potassium chloride, magnesium, a magnesium salt, or any combination thereof. In another aspect, the pharmaceutical composition further comprises an excipient or a carrier suitable to be formulated for intravenous administration, for intramuscular administration, for intrathecal administration, for intraperitoneally administration, for intrapulmonary administration, for transdermal administration, for subcutaneous administration, or for oral administration. In yet another aspect, the pharmaceutical composition comprises an injectable, a tablet, a capsule, a patch, an implant, or a depot. One aspect of the present disclosure encompasses a method of treating a glucose metabolism disorder in a subject in need thereof, comprising administering to the subject a therapeutically effective amount of the above pharmaceutical composition. In one aspect, the administering comprises delivering the leptin active to a region of the central nervous system. In one aspect, the glucose metabolic disorder is selected from the group consisting of hyperglycemia, Type I diabetes and Type II diabetes.

In any of the method and/or the pharmaceutical composition, the subject is having or being suspected of having Type I diabetes, autoimmune Type I diabetes, and/or hypoglycemia associated with autonomic failure or obesity.

Another aspect of the present disclosure encompasses a leptin compound or a modified leptin compound. The leptin compound has a sequence derived and/or modified from the amino acid sequence as set forth in SEQ ID NO.: 1. The modified leptin compound is derived from the amino acid sequence of SEQ ID NO.: 1, then modified with a moiety capable of effectuating brain uptake or accumulation and/or penetrating or permeating through the BBB.

### REFERENCE TO COLOR FIGURES

The application file contains at least one photograph executed in color. Copies of this patent application publication with color photographs will be provided by the Office upon request and payment of the necessary fee.

### BRIEF DESCRIPTION OF THE FIGURES

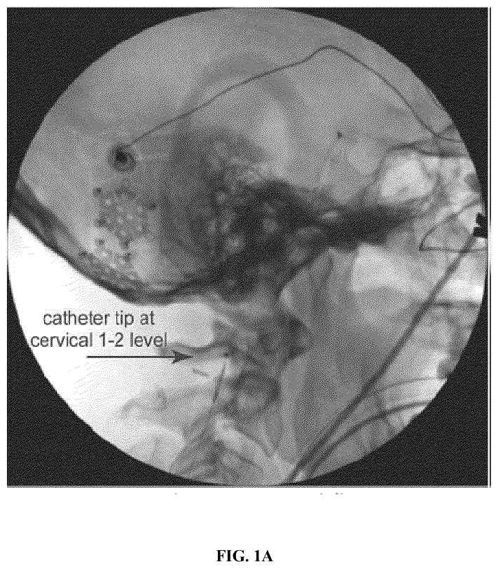

FIGS. 1A-1B are images showing intrathecal delivery according to the methods disclosed herein. FIG. 1A is a lateral craniocervical fluoroscopic image showing an intrathecal catheter with its tip, which bears a radiopaque marker, located at the C1-2 level. FIG. 1B is an image of radiopaque dye injected through the intrathecal catheter ascends via cerebrospinal fluid flow in the subarachnoid space to the region of the dorsum sellae, where it accumulates adjacent to the basal hypothalamus. Leptin delivered intrathecally via this route is therefore able to target the brain.

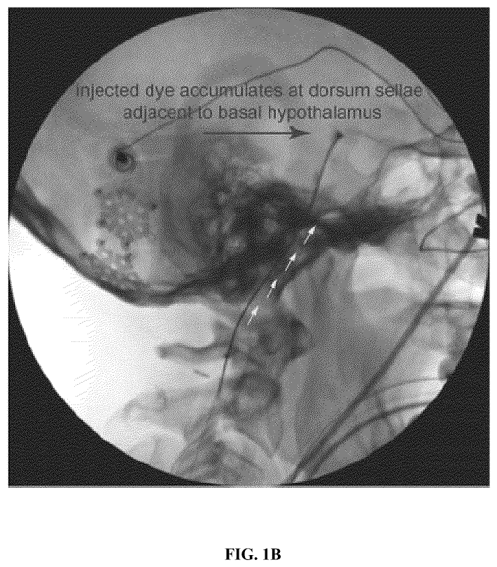

FIGS. 2A-2B depict exemplary intrathecal devices. FIG. 2A is a schematic diagram of a device from B. Braun, currently used for intrathecal medication delivery in Europe. The device has an intrathecal Port-β-Cath system for delivery of intrathecal pain medication through percutaneous injection into a subcutaneous reservoir. FIG. 2B shows two examples of a device currently available and used for intrathecal medication delivery in U.S.: Intrathecal drug infusion pumps made by Medtronic (left) and Flowonix (middle), used for continuous intrathecal drug delivery (right) in patients with chronic pain or intractable spasticity.

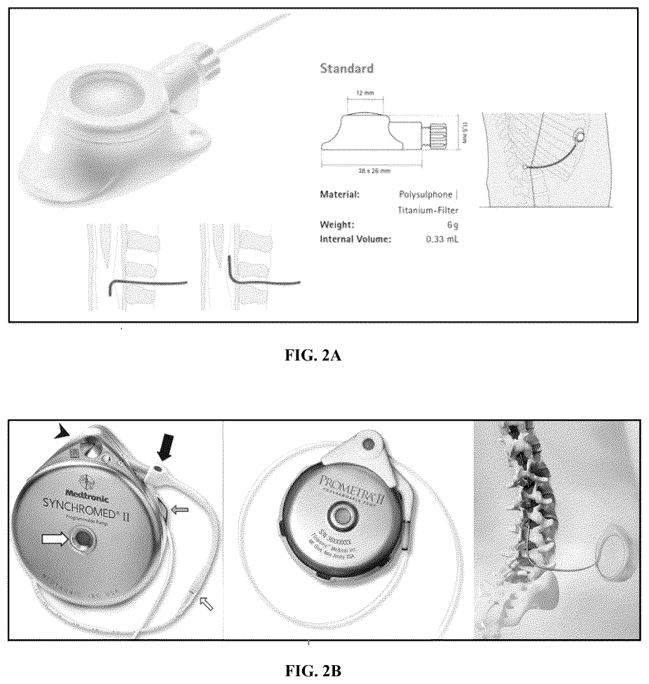

FIG. 3A is schematic diagram of the study timeline for animal studies as disclosed herein. FIG. 3B is a series of images showing intrathecal catheter placement in YF pig under general anesthesia. From left to right: catheter placement under AP and lateral fluoroscopy; CSF backflow from intrathecal catheter; confirmation of dye injection into intrathecal space in the upper cervical spine; healthy, recovered pig post-procedure.

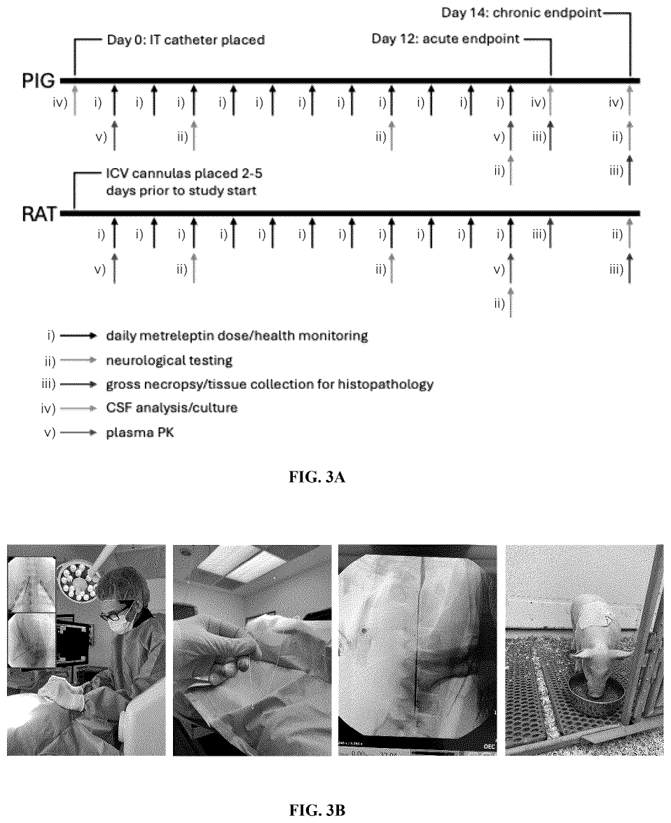

FIG. 4 is a diagram of the group assignments with safety and toxicity endpoints to be assessed following metreleptin administration directly to the brain over 11 consecutive days in Sprague-Dawley rats and Yorkshire Farm pigs.

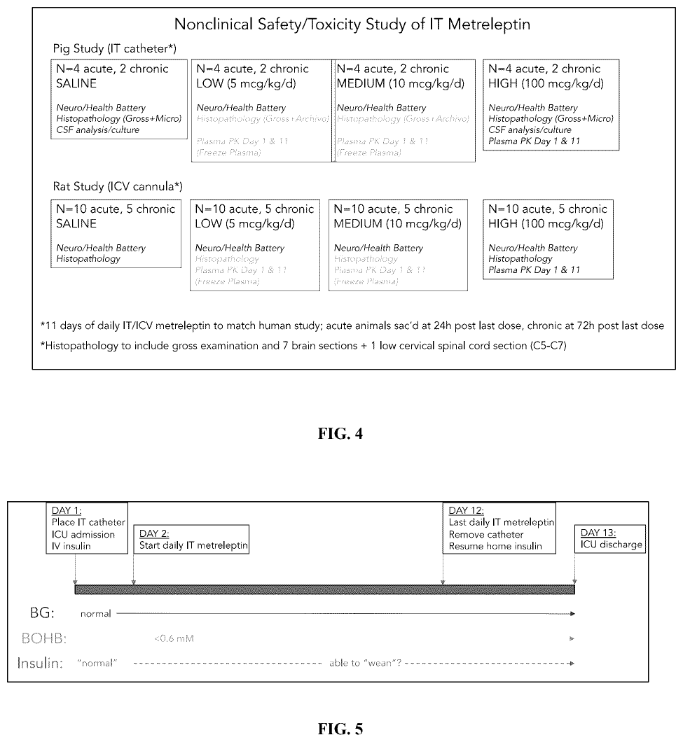

FIG. 5 is a summary diagram of an inpatient study design. Patients have the intrathecal (IT) catheter placed on day 1 and their insulin regimen is converted to an IV infusion. They then receive 11 consecutive days of a daily bolus dose of IT metreleptin, during which time their blood glucose and ketone (beta-hydroxybutryate) levels are monitored. The study is to determine whether the IV insulin infusion can be weaned as the effects of IT metreleptin begin to maintain normal glucose levels, thereby reducing insulin requirements. BG, blood glucose; BOHB, beta-hydroxybutyrate.

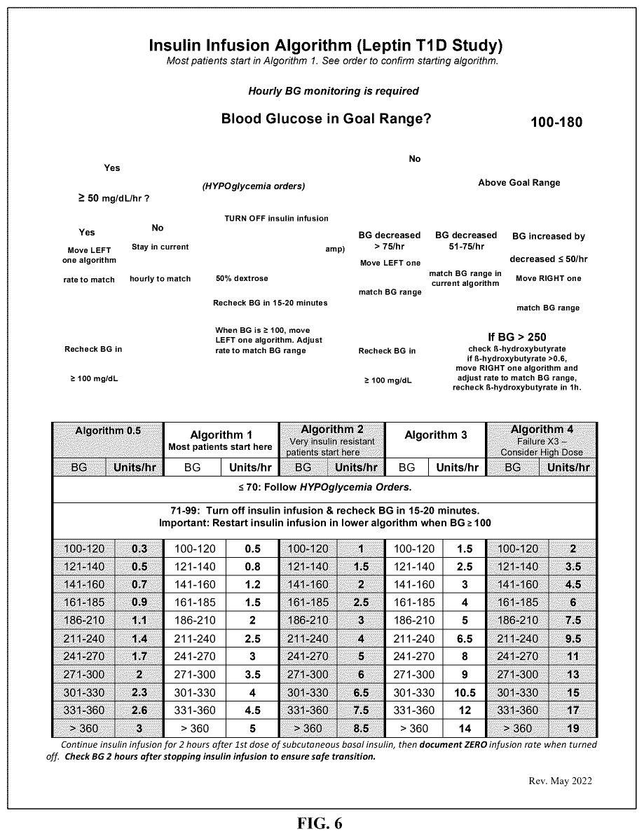

FIG. 6 depicts the insulin infusion algorithm for the in-patient clinical trial.

### DETAILED DESCRIPTION

The present disclosure is based in part on the discovery that a leptin active delivered to the central nervous system (CNS), and in particular to brain regions, is effective in treating, ameliorating, and/or preventing a glucose metabolism disorder. As used herein, the term “glucose metabolism disorder” refers to any pathological condition in which blood glucose levels cannot be maintained within the normal range. It encompasses a disease or disorder associated with abnormal insulin production, abnormal insulin sensitivity, a dysfunction of islet cells, and any combination thereof. In various aspects, a disease or disorder of glucose metabolism is selected from or encompasses hyperglycemia, Type I diabetes, and Type II diabetes, including subtypes and related disorders all characterized by hyperglycemia and glucose intolerance, such as Wolfram Syndrome, Lipoatrophic Diabetes, Gestational Diabetes, Donohue Syndrome, Glycosuria, Hypeinsulinism, Insulin Resistance, Insulin Coma, or Latent Autoimmune Diabetes.

The leptin active as described herein encompasses a leptin compound, a modified leptin compound, a pharmaceutically acceptable salt thereof, a crystal form thereof, an enantiomer thereof, or any combination thereof. The leptin compound comprises a leptin, a leptin isomer, a leptin derivative, a leptin analog, a leptin mimetic, a leptin agonist, an anti-leptin receptor antibody, or any combination thereof. A modified leptin compound is a leptin compound further modified by a moiety capable of effectuating and/or enhancing brain uptake of the leptin compound from the bloodstream following systemic delivery. The therapeutic and/or prophylactic effect of the leptin active is independent of insulin or blood glucose level, therefore avoiding various drawbacks and shortcomings of the present standard of care treatment of diabetes or hyperglycemia. The ability of the treatment methods and compositions disclosed herein to target the brain to normalize blood sugar in T1D patients is unprecedented. Establishing the brain's ability to control blood glucose without the need for insulin opens a new chapter in the history of diabetes and has the potential to provide tremendous benefit to many patients.

### I. Leptin Active

The present disclosure relates to a leptin active, and therapeutic and/or prophylactic use of such leptin active. For most animal species, a naturally occurring leptin is a 16-kDa pepT1De hormone secreted from white adipocytes. It is structurally related to cytokines and acts on receptors that belong to the cytokine receptor superfamily. It serves as a feedback signal from fat cells to the central nervous system in regulation of food intake, energy balance, and fat storage. As used herein, a leptin active refers to any therapeutic or prophylactic active developed based on a naturally occurring leptin, such as the one from human or the one from a mammal. In one aspect, the leptin active encompasses a leptin compound, a modified leptin compound, or any combination thereof. The leptin compound may comprise or be selected from a leptin, a leptin isomer, a leptin derivative, a leptin analog, a leptin mimetic, a leptin agonist, an anti-leptin receptor antibody, or any combination thereof. As used herein, leptin refers to any naturally occurring leptin, such as those isolated from a human being or from a mammal. A leptin isomer can be a positional isomer or a configuration isomer, such as the trans-10, cis-12 isomer or the cis-9, trans-11 isomer. The present disclosure encompasses all possible leptin isomers. A leptin derivative refers to a pepT1De or a pepT1Domimetic based on a naturally occurring leptin sequence but is derivatized at various positions with chemical bonds and/or functional groups. Such leptin derivative preserves the biological function of the naturally occurring leptin. A leptin analog refers to any chemical entity that functions similarly as a naturally occurring leptin but is structurally different from the natural occurring leptin. A leptin mimetic has a sequence similar to a naturally occurring leptin and possesses similar functions. A modified leptin compound refers to a leptin compound further modified by a moiety capable of effectuating and/or enhancing brain uptake of the leptin compound. One example of such modified leptin compound is a conjugate formed by attaching a Blood-Brain-Barrier (BBB) penetrating moiety to the leptin compound. BBB penetrating moiety comprises at least one fragment selected from the group consisting of a transferrin receptor, an insulin receptor, an insulin-like growth factor receptor, a low density lipoprotein receptor, a low density lipoprotein receptor-related protein 8, a low density lipoprotein receptor-related protein 1, a heparin-binding epidermal growth factor-like growth factor, an antibody thereof, or any combination thereof. The leptin active also includes all pharmaceutically acceptable salts, crystal forms, and enantiomers thereof,

In one aspect, the present disclosure encompasses metreleptin as the leptin active. Metreleptin is a recombinant human leptin analog composed of human leptin with the addition of a methionine residue at its amino terminus. Metreleptin has an amino acid sequence set forth in SEQ ID NO:1. In one aspect, the present disclosure encompasses a leptin active derived from the amino acid sequence of SEQ ID NO:1. In some aspects, the leptin active comprises an amino acid sequence having at least about 80%, 85%, 90%, 95%, or more sequence identity to the amino acid sequence set forth in SEQ ID NO:1. In some aspects, the leptin active comprises the amino acid sequence set forth in SEQ ID NO:1 comprising one or more amino acid substitutions. In some aspects, the leptin active comprises the amino acid sequence set forth in SEQ ID NO:1.

In another aspect, the present disclosure encompasses a leptin active derived from the amino acid sequence set forth in SEQ ID NO:1, then modified with a moiety capable of effectuating brain uptake or accumulation and/or penetrating or permeating through the BBB. MYALEPT is the brand name for metreleptin formulated for subcutaneous injections and is commercially available and indicated as an adjunct to diet as replacement therapy to treat the complications of leptin deficiency in patients with congenital or acquired generalized lipodystrophy.

### II. Treatment

The present disclosure relates to treating, ameliorating, and/or preventing a glucose metabolism disorder as disclosed herein. As used herein, the term “glucose metabolism disorder” refers to any pathological condition in which the blood glucose cannot be maintained within the normal range. It encompasses a disease or disorder associated with abnormal insulin production, abnormal insulin sensitivity, a dysfunction of islet cells, and any combination thereof. In various aspects, a disease or disorder of glucose metabolism is selected from or encompasses hyperglycemia, Type I diabetes, and Type II diabetes, including subtypes and related disorders all characterized by hyperglycemia and glucose intolerance, such as Wolfram Syndrome, Lipoatrophic Diabetes, Gestational Diabetes, Donohue Syndrome, Glycosuria, Hypeinsulinism, Insulin Resistance, Insulin Coma, or Latent Autoimmune Diabetes. Since blood glucose concentration is critical to survival by being the predominant fuel for the central nervous system, the glucose metabolism disorder may manifest other systemic diseases or disorders. In one aspect, the glucose metabolism disorder is Type I diabetes (T1D), including sub-types, such as autoimmune T1D, or hypoglycemia associated with autonomic failure or obesity.

In some aspects, provided herein is a method of treating a glucose metabolism disorder in a subject in need thereof, the method comprising: administering to the subject a therapeutically effective amount of a leptin active, wherein the administering comprises delivering the leptin active to the central nervous system.

### Patient Populations

The methods provided herein are useful for treatment of a range of subjects having a glucose metabolism disorder. For example, in some aspects, the glucose metabolism disorder is hyperglycemia, Type I diabetes, or Type II diabetes. In other aspects, the subject has Wolfram Syndrome, Lipoatrophic Diabetes, Gestational Diabetes, Donohue Syndrome, Glycosuria, Hypeinsulinism, Insulin Resistance, Insulin Coma, or Latent Autoimmune Diabetes.

In some aspects, the subject is a male. In other aspects, the subject is a female. In some aspects, the subject is between the ages of about 18 to about 80, such as about 18 to about 75, about 18 to about 70, about 18 to about 65, about 18 to about 60, about 18 to about 55, about 18 to about 50, about 18 to about 45, or about 18 to about 40 years of age. In some aspects, the subject is between the ages of about 18 to about 40.

In some aspects, the subject is a male. In other aspects, the subject is a female. In some aspects, the subject is between the ages of 18 to 80, such as about 18 to 75, 18 to 70, 18 to 65, 18 to 60, 18 to 55, 18 to 50, 18 to 45, or 18 to 40 years of age. In some aspects, the subject is between the ages of 18 to 40.

In some aspects, the subject is a non-smoker with no current indication of drug or alcohol abuse.

In some aspects, the subject has been diagnosed with a glucose metabolism disorder for at least one year. In some aspects, the subject has been diagnosed with Type I diabetes for at least about 1 year. In some aspects, the diagnosis of Type I diabetes is based on clinical criteria including, but not limited to, insulin-dependence within 6 months of the onset, history of prior episode of ketoacidosis, previous documentation of positive serum diabetes autoantibodies such as GAD, IA-2, or Zinc transporter or low or undetectable serum C-pepT1De levels.

In some aspects, the subject has an HbA1c level of less than about 7.0% or less than 7.0%.

In other aspects, the subject has a BMI of between about 20 and 27 kg/m², or between 20 and 27 kg/m².

In some aspects, the subject is currently treated by subcutaneous insulin pump and continuous glucose monitor (CGM). In some aspects, the subject is currently treated with automated insulin delivery.

In some aspects, the subject does not have diabetic complications such as clinical neuropathy or evidence of autonomic dysfunction or nephropathy.

In some aspects, the subject is not taking systemic glucocorticoids or sex steroids. In other aspects, the subject is not taking sympatholytic or sympathomimetic agents.

In some aspects, the subject does not have a malignant neoplasm.

### End Points

The methods provided herein are useful for treating, ameliorating, and/or preventing a glucose metabolism disorder. In some aspects, the methods provided herein normalize blood glucose levels in a subject with Type 1 diabetes without insulin therapy. In some aspects, the methods provided herein result in a fasting blood glucose level of between about 70 and about 125 mg/dL, or between 70 and 125 mg/dL, in the subject. In other aspects, the methods provided herein result is a beta hydroxybutyrate level of less than about 0.6 mM, or less than 0.6 mM, during an inpatient treatment period of up to 14 days.

In other aspects, the methods provided herein achieve an improved CGM time in range (TIR) percentage compared to existing treatments. For example, the methods provided herein achieve a 50%, a 60%, a 70%, an 80% or a 90% CGM TIR percentage for at least about three days, or about a 50%, about a 60%, about a 70%, about an 80% or about a 90% CGM TIR percentage for at least about three days. In some aspects, the in-range blood glucose level is calculated as a blood glucose level of between 70 mg/dL to 180 mg/dL, or between about 70 mg/dL to about 180 mg/dL.

In other aspects, the methods provided herein achieve a reduced CGM time-above-range (TIHyper^{CGM}) percentage compared to existing treatments. For example, the methods provided herein achieve a 5%, a 10%, a 15%, a 20% or a 25% TIHyper^{CGM} percentage for at least three days. In other aspects, the methods provided herein achieve about a 5%, about a 10%, about a 15%, about a 20% or about a 25% TIHyper^{CGM} percentage for at least about three days. In some aspects, the above-range blood glucose level is calculated as a blood glucose level of above about 180 mg/dL or above 180 mg/dL.

In some aspects, the methods provided herein achieve a reduced CGM time-below-range (TIHypo^{CGM}) percentage compared to existing treatments. For example, the methods provided herein achieve less than a 5%, a 10%, a 15%, a 20% or a 25% TIHypo^{CGM} percentage for at least three days. In other aspects, the methods provided herein achieve less than about a 5%, about a 10%, about a 15%, about a 20% or about a 25% TIHypo^{CGM} percentage for at least about three days. In some aspects, the below range blood glucose level is calculated as a blood glucose level of below 70 mg/dL or about 70 mg/dL.

In some aspects, the methods provided herein achieve an improved oral glucose tolerance test (OGTT) outcome compared to existing treatments. In some aspects, the OGTT outcome is calculated as a blood glucose level of less than about 140 mg/dL three hours following the initiation of the OGTT, or less than 140 mg/dL three hours following the initiation of the OGTT.

In some aspects, the methods provided herein achieve improved levels of food intake in the subject after treatment compared to the level of food intake in the subject prior to treatment compared to existing treatments.

In other aspects, the methods provided herein achieve improved levels of insulin dose in the subject after treatment compared to the levels of insulin dose in the subject prior to treatment compared to existing treatments.

In other aspects, the methods provided herein achieve improved levels of plasma counterregulatory hormones in the subject after treatment compared to the levels of plasma counterregulatory hormones in the subject prior to treatment compared to existing treatments. In some aspects, the plasma counterregulatory hormones comprise glucagon, cortisol, epinephrine, or norepinephrine.

In other aspects, the methods provided herein achieve improved levels of lipids, triglycerides, and/or free fatty acids in the subject after treatment compared to the levels of lipids, triglycerides, and/or free fatty acids in the subject prior to treatment compared to existing treatments.

In other aspects, the methods provided herein achieve improved levels of plasma leptin in the subject after treatment compared to the levels of plasma leptin in the subject prior to treatment compared to existing treatments.

### III. Blood Brain Barrier

The present disclosure encompasses compositions of matter, compositions, methods, means and/or practices to effectuate the delivery of a therapeutic agent to the brain following a systemic delivery, by allowing the therapeutic agent to penetrate and/or permeate effectively through the blood-brain barrier (BBB), and/or to allow uptake and accumulation in the brain. The high selectivity and limited permeability of the BBB serves an important protective function, but at the same time prevents entry of large biotherapeutic or a small molecule from the bloodstream. Strategies to overcome this obstacle have been described and include various methods and means to effectuate the delivery of a therapeutic agent to the brain. These include but are not limited to: (1) using a physical device to deliver the therapeutic agent locally to the central nervous system; (2) applying an external force to temporarily disrupt BBB to facilitate delivery of the therapeutic agent to the brain; (3) formulating a composition capable of allowing the therapeutic agent to penetrate and/or permeate through the BBB, or improve the penetration efficiency; (4) modifying the therapeutic agent with a BBB penetrating moiety to improve the BBB penetration and/or permeation; or (5) any combination thereof.

In one aspect, the methods and/or means to facilitate brain delivery comprises applying an external force to disrupt BBB. In one aspect, such external force comprises an acoustic radiation force and/or an acoustic cavitation force to temporarily disrupt BBB. Such acoustic force may be in the form of an ultrasound, a focused ultrasound (FUS), and optionally concurrently with systemically administered microbubbles. In one aspect, the ultrasound is a 220 kHz FUS transducer in combination with microbubbles. In another aspect, the subject being treated is treated with the BBB opening sessions before and/or while being systemically administered the leptin active.

Compositions to facilitate brain delivery can maximize brain uptake and/or accumu In some aspects, the subject is a male. In other aspects, the subject is a female. In some aspects, the subject is between the ages of about 18 to about 80, such as about 18 to about 75, about 18 to about 70, about 18 to about 65, about 18 to about 60, about 18 to about 55, about 18 to about 50, about 18 to about 45, or about 18 to about 40 years of age. In some aspects, the subject is between the ages of about 18 to about 40 years of age, and these include but are not limited to a hypertonic osmotic agent, such as mannitol, glycerol, lactulose, sorbitol, or polyethylene glycol. In another aspect, the osmotic agent comprises an osmogenic agent, such as sodium chloride, potassium chloride, and a sugar. In another aspect, the composition comprises magnesium or magnesium salt. In another aspect, the magnesium salt comprises both a magnesium organic salt and/or a magnesium inorganic salt. In another aspect, the composition comprises mannitol and magnesium.

In one aspect, the methods and/or means is to utilize transcytosis pathways mediated by endogenous receptors expressed on the brain capillary endothelium (blood brain barrier receptor). In one aspect, the composition comprises an endogenous receptor capable of facilitating BBB shuttle comprises a transferrin receptor, an insulin receptor, an insulin-like growth factor receptor, a low density lipoprotein receptor, a low density lipoprotein receptor-related protein 8, a low density lipoprotein receptor-related protein 1, a heparin-binding epidermal growth factor-like growth factor, or any combination thereof. In another aspect, the composition comprises a recombinant protein designed against the endogenous receptor to enable receptor-mediated brain delivery of the therapeutic agents. Such recombinant protein may be in the form of an antibody or a pepT1De. In one aspect, the antibody comprises a monoclonal antibody, a polyclonal antibody, or a recombinant antibody.

Compositions as disclosed herein encompass a therapeutic agent in the form of a leptin compound modified with a BBB penetrating moiety, wherein the modified leptin compound possesses an improved BBB penetration and/or permeation ability, when compared with the unmodified leptin compound. Various fat-soluble substances may serve as a BBB penetrating moiety that is capable of modifying and improving the lipophilicity of the leptin compound. In one aspect, the BBB penetrating moiety comprises a fragment or multiple fragments from a BBB shuttle molecule. In one aspect, the BBB shuttle molecule comprises a transferrin receptor, an insulin receptor, an insulin-like growth factor receptor, a low-density lipoprotein receptor, a low-density lipoprotein receptor-related protein 8, a low-density lipoprotein receptor-related protein 1, a heparin-binding epidermal growth factor-like growth factor, an antibody thereof, or any combination thereof.

### IV. Drug Delivery Devices

One aspect of the present disclosure encompasses a method of delivering the leptin active to the central nervous system (CNS). The CNS delivery may be made to a region of the brain, to a region of the spinal cord, or to a region of the meninges. In one aspect, the brain region is selected from the hippocampus, frontal cortex, parietal cortex, or a region or regions within any one or more of same. In another aspect, the brain region is selected from one or more regions of the hypothalamus, or the arcuate nucleus, ventromedial nucleus of the hypothalamus, and the nucleus of the solitary tract of the hindbrain.

In other aspects the CNS delivery comprises delivering to a region of the spinal cord, including but not limited to the cervical spine, lumbar spine, and/or the intervertebral spaces. In one aspect, the delivery is from the lumbar 2-3 level to the cervical 2 level.

In another aspect, delivery involves using a physical device or apparatus such as a catheter, a reservoir, or a pump. In the present disclosure, a catheter can be designed or repurposed to deliver a therapeutic agent locally to CNS or targeted region or regions within the CNS.

In several aspects of the present disclosure, a drug delivery device as disclosed herein is capable of enhancing delivery of the leptin active to the brain or a targeted brain region by using implanted subcutaneous or transcutaneous reservoirs or port devices. These reservoirs or devices are connected at the proximal end of the intrathecal catheter, whereby the leptin compound is administered percutaneously or transcutaneously. Such set up aids in the delivery of the leptin active to the cerebrospinal fluid via the intrathecal catheter. These devices include reservoirs that store the leptin active subcutaneously for a period of time prior to administration through the catheter. Alternatively, the delivery can be achieved by implanted subcutaneous automated infusion pumps. Such pumps deliver the leptin active continuously or in a scheduled manner, such as a daily bolus dose, over a period of time with percutaneous refills of the pump. Another option is to use an intrathecal catheter with a distal aperture design that improves rostral flow of the leptin active in the cerebrospinal fluid for targeted brain delivery.

In one aspect, a delivery device is or includes an intrathecal drug infusion pump. Various such infusion pumps are known, such as the Ascenda™ catheter made by and available from Medtronic. Other suitable devices are manufactured by Flowonix or B. Braun. A device commercially available from B. Braun is widely used in Europe for intrathecal medication delivery. The device has an intrathecal Port-a-Cath system for delivery of intrathecal pain medication through percutaneous injection into a subcutaneous reservoir. Other options are the SynchroMed™ II-Drug Infusion Systems made by Medtronic or Prometra pump from Flowonix II. It should be understood that the methods described herein contemplate delivery using any device designed specifically for delivering an active agent to the brain.

### V. Systems or Kits for Medical Use

One aspect of the present disclosure encompasses a kit or a system having one or more functions of monitoring, evaluating, or therapy delivering to a subject having or is suspect of having a metabolic disease. In one aspect, the metabolic disorder may be hyperglycemia, a disorder involving glucose metabolism, a disorder involving abnormal insulin production, a disorder involving abnormal insulin sensitivity, a dysfunction of islet cells, or any combination thereof. In one aspect, the subject is having, is suspect of having, or having a high risk of contracting hyperglycemia, Type I diabetes or Type II diabetes. In one aspect, the kit or system may comprise comprising (i) a measuring device for measuring and recording blood glucose or insulin levels of the subject; (ii) a communication device for relaying the recorded levels to a physician; and (iii) a monitoring device accessible by the physician for monitoring the relayed levels and proposing the physician with a treatment plan. In one aspect, the treatment plan comprises administering to the subject a therapeutically effective amount of the leptin compound or the modified leptin compound. In another aspect, such administering effectuates delivery of the leptin compound and/or the modified leptin compound to a brain region.

### VI. Pharmaceutical Compositions

One aspect of the disclosure encompasses a pharmaceutical composition comprising the leptin active. The pharmaceutical composition is formulated to deliver a therapeutically effective amount of the leptin active to the site of action. In various aspects of the disclosure, the site of action is the brain. Specifically, it may include one or more regions of the brain, such as the arcuate nucleus, the ventromedial nucleus of the hypothalamus, or the nucleus of the solitary tract of the hindbrain. In another aspect, the therapeutically effective amount of the leptin active is from about 5.0 mcg/kg/day to about 10.0 mcg/kg/day. In various aspects, the leptin active or the composition comprising the leptin active is administered to the subject once daily, once every two days, once every three days, once every four days, once every five days, once every six days, weekly or monthly. In various other aspects, the administrative period may be indefinite or long term, but can also start with a short term, such as at least 5 days, at least 6 days, least 7 days, at least 8 days, at least 9 days, at least 10 days, at least 11 days, or at least 12 days. In one aspect, the pharmaceutical composition is suitable for local delivery. In another aspect, the pharmaceutical composition is for systemic delivery, such as through intravenous injection, intramuscular injection, subcutaneous injection, transdermal delivery, buccal delivery, or oral delivery. In one aspect, the pharmaceutical composition comprises an endogenous receptor capable of facilitating BBB shuttle. In another aspect, the endogenous receptor comprises a transferrin receptor, an insulin receptor, an insulin-like growth factor receptor, a low-density lipoprotein receptor, a low-density lipoprotein receptor-related protein 8, a low-density lipoprotein receptor-related protein 1, a heparin-binding epidermal growth factor-like growth factor, or any combination thereof. In another aspect, the BBB shuttle may be facilitated by having an excipient in the form of a recombinant protein designed against the endogenous receptor to enable receptor-mediated brain delivery of the therapeutic agents. Such recombinant protein may be in the form of an antibody or a pepT1De. In one aspect, the antibody comprises a monoclonal antibody, a polyclonal antibody, or a recombinant antibody. In another aspect, the composition comprises the modified leptin wherein the leptin compound is conjugated with a BBB penetrating moiety. In one aspect, the BBB penetrating moiety comprises at least one fragment selected from the group consisting of a transferrin receptor, an insulin receptor, an insulin-like growth factor receptor, a low density lipoprotein receptor, a low density lipoprotein receptor-related protein 8, a low density lipoprotein receptor-related protein 1, a heparin-binding epidermal growth factor-like growth factor, an antibody thereof, or any combination thereof.

The present disclosure encompasses a pharmaceutical composition further comprise at least one excipient or carrier selected from the group consisting of an osmotic agent, an osmogenic agent, a sugar, an inorganic salt, and any combination thereof. Specifically, the at least one excipient may comprise mannitol, glycerol, lactulose, sorbitol, polyethylene glycol, glutamic acid, glycine, polysorbate 20, sucrose, sodium chloride, potassium chloride, magnesium, a magnesium salt, or any combination thereof.

The present disclosure encompasses a pharmaceutical composition comprises a therapeutically effective amount of the leptin active in freebase form, in a salt form, in an amorphous form, in a crystal form, or in a racemic form. The salt form is a pharmaceutically acceptable salt, include, without limitation, acetate, aspartate, benzoate, bitartrate, citrate, formate, gluconate, glucuronate, glutamate, fumarate, hydrochloride, hydrobromide, hydroiodide, hypophosphite, isobutyrate, isocitrate, lactate, malate, maleate, meconate, methylbromide, methanesulfonate, monohydrate, mucate, nitrate, oxalate, phenylpropionate, phosphate, phthalate, propionate, pyruvate, salicylate, stearate, succinate, sulfate, tannate, tartrate, terephthalate, valerate, and the like.

The leptin active can be formulated and administered to the subject by several different means. For instance, a pharmaceutical composition comprising the leptin active can be administered parenterally, intraperitoneally, intravascularly, transdermally, subcutaneously, or intrapulmonarily in dosage unit formulations containing conventional nontoxic pharmaceutically acceptable adjuvants, carriers, excipients, and vehicles as desired. The term parenteral as used herein includes subcutaneous, intravenous, intramuscular, intrathecal, or intrasternal injection, or infusion techniques. Formulation of pharmaceutical compositions is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

A pharmaceutical composition also comprises one or more pharmaceutically acceptable excipients. Non-limiting examples of excipients include chemical enhancers, humectants, pressure sensitive adhesives, antioxidants, solubilizers, thickening agents, plasticizers, adjuvants, carriers, excipients, vehicles, coatings, and any combinations thereof. One or more excipients can be selected for oral, transdermal, parenteral, intraperitoneal, intravascular, subcutaneous, by inhalation spray, rectal, or intrapulmonary administration.

The leptin active can in general be formulated for improving patient compliance, preventing a subject from removing the drug-delivery device. For instance, the leptin active could be formulated for improved patient compliance and preventing removal of a drug-delivery device by providing formulations for extended delivery. Extended delivery can range for periods ranging from more than one day, to months. This may be especially relevant for patients with compromised cognitive and/or motor-control abilities. Extended delivery for periods can range from about 1 day to about 1 year, from about 1 day to about 1 week, from about 3 days to about 1 month, from about 2 weeks to about 6 months, or from about 2 months to about 4 months.

Extended-release formulations could be used for substantially continuous delivery of the leptin active at a preselected rate. For example, the leptin active can be delivered at about 5.0 mcg/kg/day to about 10.0 mcg/kg/day. Appropriate amounts of the leptin active can be readily determined by the ordinarily skilled artisan based upon, for example, the intended duration of administration of the leptin active by the extended-release formulation, the delivery mechanism, the particular formulation, and the relative potency of the drug among other factors.

### Binders

Non-limiting examples of binders suitable for the formulations of various aspects include starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohols, polyethylene glycol, polyols, saccharides, oligosaccharides, polypepT1Des, oligopepT1Des, and combinations thereof. The polypepT1 De may be any arrangement of amino acids ranging from about 100 to about 300,000 Daltons.

The binder can be introduced into the mixture to be granulated in a solid form, including but not limited to a crystal, a particle, a powder, or any other finely divided solid form known in the art. Alternatively, the binder can be dissolved or suspended in a solvent and sprayed onto the mixture in a granulation device as a binder fluid during granulation.

### Diluents

Non-limiting examples of diluents (also referred to as “fillers” or “thinners”) include carbohydrates, inorganic compounds, and biocompatible polymers, such as polyvinylpyrrolidone (PVP). Other non-limiting examples of diluents include dibasic calcium sulfate, tribasic calcium sulfate, starch, calcium carbonate, magnesium carbonate, microcrystalline cellulose, dibasic calcium phosphate, tribasic calcium phosphate, magnesium carbonate, magnesium oxide, calcium silicate, talc, modified starches, saccharides such as sucrose, dextrose, lactose, microcrystalline cellulose, fructose, xylitol, and sorbitol, polyhydric alcohols; starches; pre-manufactured direct compression diluents; and mixtures of any of the foregoing.

### Disintegrants

Disintegrents can be effervescent or non-effervescent. Non-limiting examples of non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Suitable effervescent disintegrants include but are not limited to sodium bicarbonate in combination with citric acid, and sodium bicarbonate in combination with tartaric acid.

### Preservatives

Non-limiting examples of preservatives include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apo-carotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphaT1 Dylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanilic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivates, vitamin Q10, wheat germ oil, zeaxanthin, or combinations thereof.

### Flavor-Modifying Agents

Suitable flavor-modifying agents include flavorants, taste-masking agents, sweeteners, and the like. Flavorants include, but are not limited to, synthetic flavor oils and flavoring aromatics and/or natural oils, extracts from plants, leaves, flowers, fruits, and combinations thereof. Other non-limiting examples of flavors include cinnamon oils, oil of wintergreen, peppermint oils, clover oil, hay oil, anise oil, eucalyptus, vanilla, citrus oils such as lemon oil, orange oil, grape and grapefruit oil, fruit essences including apple, peach, pear, strawberry, raspberry, cherry, plum, pineapple, and apricot.

Taste-masking agents include but are not limited to cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other aspects, additional taste-masking agents contemplated are those described in U.S. Pat. Nos. 4,851,226; 5,075,114; and 5,876,759, each of which is hereby incorporated by reference in its entirety.

Non-limiting examples of sweeteners include glucose (corn syrup), dextrose, invert sugar, fructose, and mixtures thereof (when not used as a carrier); saccharin and its various salts such as the sodium salt; dipepT1De sweeteners such as aspartame; dihydrochalcone compounds, glycyrrhizin; Stevia rebaudiana (Stevioside); chloro derivatives of sucrose such as sucralose; sugar alcohols such as sorbitol, mannitol, sylitol, hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

### Lubricants and Glidants

The lubricant compositions may be utilized to lubricate ingredients that form a pharmaceutical composition. As a glidant, the lubricant facilitates removal of solid dosage forms during the manufacturing process. Non-limiting examples of lubricants and glidants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. The pharmaceutical composition will generally comprise from about 0.01% to about 10% by weight of a lubricant. In some aspects, the pharmaceutical composition will comprise from about 0.1% to about 5% by weight of a lubricant. In a further aspect, the pharmaceutical composition will comprise from about 0.5% to about 2% by weight of a lubricant.

### Dispersants

Dispersants may include but are not limited to starch, alginic acid, polyvinylpyrrolidones, guar gum, kaolin, bentonite, purified wood cellulose, sodium starch glycolate, isoamorphous silicate, and microcrystalline cellulose as high hydrophilic-lipophilic balance (HLB) emulsifier surfactants.

### Colorants

Depending upon the aspect of the disclosure, it may be desirable to include a coloring agent. Suitable color additives include but are not limited to food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in various aspects of the disclosure.

### pH Modifiers

Non-limiting examples of pH modifiers include citric acid, acetic acid, tartaric acid, malic acid, fumaric acid, lactic acid, phosphoric acid, sorbic acid, benzoic acid, sodium carbonate and sodium bicarbonate.

### Chelating Agents

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na2EDTA).

### Antimicrobial Agents

An antimicrobial agent may be included as an excipient to minimize the degradation of the compound according to this disclosure by microbial agents, including but not limited to bacteria and fungi. Non-limiting examples of antimicrobials include parabens, chlorobutanol, phenol, calcium propionate, sodium nitrate, sodium nitrite, Na2EDTA, and sulfites including but not limited to sulfur dioxide, sodium bisulfite, and potassium hydrogen sulfite.

### Release-Controlling Polymers

Release-controlling polymers may be included in the various aspects of the solid dosage pharmaceutical compositions incorporating compounds according to this disclosure. In one aspect, the release-controlling polymers may be used as a tablet coating. In other aspects, including but not limited to bilayer tablets, a release-controlling polymer may be mixed with the granules and other excipients prior to the formation of a tablet by a known process including but not limited to compression in a tablet mold. Suitable release-controlling polymers include but are not limited to hydrophilic polymers and hydrophobic polymers.

Suitable hydrophilic release-controlling polymers include, but are not limited to, cellulose acetate, cellulose diacetate, cellulose triacetate, cellulose ethers, hydroxyethyl cellulose, hydroxypropyl cellulose, hydroxypropyl methylcellulose, microcrystalline cellulose, nitrocellulose, crosslinked starch, agar, casein, chitin, collagen, gelatin, maltose, mannitol, maltodextrin, pectin, pullulan, sorbitol, xylitol, polysaccharides, ammonia alginate, sodium alginate, calcium alginate, potassium alginate, propylene glycol alginate, alginate sodium carmellose, calcium carmellose, carrageenan, fucoidan, furcellaran, arabic gum, carrageens gum, ghafti gum, guar gum, karaya gum, locust bean gum, okra gum, tragacanth gum, scleroglucan gum, xanthan gum, hypnea, laminaran, acrylic polymers, acrylate polymers, carboxyvinyl polymers, copolymers of maleic anhydride and styrene, copolymers of maleic anhydride and ethylene, copolymers of maleic anhydride propylene or copolymers of maleic anhydride isobutylene), crosslinked polyvinyl alcohol and poly N-vinyl-2-pyrrolidone, diesters of polyglucan, polyacrylamides, polyacrylic acid, polyamides, polyethylene glycols, polyethylene oxides, poly(hydroxyalkyl methacrylate), polyvinyl acetate, polyvinyl alcohol, polyvinyl chloride, polystyrenes, polyvinylpyrrolidone, anionic and cationic hydrogels, and combinations thereof.

### Coatings

A solid dosage comprising a compound according to this disclosure may comprise a coating, wherein such a coating may control release of the compound, act as a moisture barrier, or buffer or modify pH. A “control releasing coat” or “controlled release coat” as used herein is defined to mean a functional coat which can for example comprise at least one pH independent polymer, pH dependent polymer (for example enteric or reverse enteric type polymers), soluble polymer, insoluble polymer, lipids, lipidic materials, or combinations thereof. The coating, when applied onto a dosage form, may slow (for example when applied to a normal release matrix dosage form), further slow (for example when applied to a controlled release matrix dosage form) or modify the rate of release of a compound according to this disclosure when applied to an uncoated dosage form. For example, the control releasing coat can be designed such that when the control releasing coat is applied to a dosage form, the dosage form in conjunction with the control releasing coat can exhibit the release of the compound according to this disclosure, such as a “modified-release”, “controlled-release”, “sustained-release”, “extended-release”, “delayed-release”, “prolonged-release,” or combinations thereof. The “control releasing coat” may optionally comprise additional materials that may alter the functionality of the control releasing coat.

The term “moisture barrier” as used herein is one which impedes or retards the absorption of moisture. Compounds according to this disclosure may be hygroscopic and, as such, may be susceptible to decomposition over time under highly humid conditions. The proportion of the components of the moisture barrier and the amount of the moisture barrier optionally applied onto the control-releasing coating or onto the core are typically such that the moisture barrier does not fall within the USP definition and requirement for an enteric coat. Suitably, the moisture barrier may comprise an enteric and/or acrylic polymer, suitably an acrylic polymer, optionally a plasticizer, and a permeation enhancer. The permeation enhancer is a hydrophilic substance, which allows water to enter without physical disruption of the coating. The moisture barrier may additionally comprise other conventional inert excipients, which may improve processing of an extended-release formulation.

Coating and matrix materials which may be used in accordance with the present disclosure are those known in the art for use in controlled-release formulations, such as synthetic polymers of the polyvinyl type, e.g., polyvinylchloride, polyvinylacetate and copolymers thereof, polyvinylalcohol, and polyvinylpyrrolidone; synthetic polymers of the polyethylene type, e.g., polyethylene and polystyrene; acrylic acid polymers; biopolymers or modified biopolymers, such as cellulosic polymers, shellac and gelatin; fats, oils, higher fatty acids and higher alcohols (i.e., acids and alcohols containing alkyl chains of at least 10 carbon atoms), for example aluminum monostearate, cetylalcohol, hydrogenated beef tallow, hydrogenated castor oil, 12-hydroxystearl alcohol, glyceryl mono- or dipalmitate; glyceryl mono-, di- or tristearate; myristyl alcohol, stearic acid, stearyl alcohol, and polyethyleneglycols; waxes; sugars and sugar alcohols.

The pH-buffering properties of a coating may be strengthened by introducing into the coating substances chosen from a group of compounds usually used in antacid formulations, for example magnesium oxide, hydroxide or carbonate, aluminum or calcium hydroxide, carbonate or silicate; composite aluminum/magnesium compounds, for example Al2O3·6MgO·CO2·12H2O, (Mg6Al2(OH)16CO3·4H2O), MgO·Al2O3.2·SiO2·nH2O, aluminum bicarbonate coprecipitate or similar compounds; or other pharmaceutically acceptable pH-buffering compounds, for example the sodium, potassium, calcium, magnesium and aluminum salts of phosphoric, carbonic, citric or other suitable, weak, inorganic or organic acids; or suitable organic bases, including basic amino acids; and salts or combinations thereof.

A pH-dependent coating serves to release the drug in desired areas of the gastrointestinal (GI) tract, e.g., the stomach or small intestine. When a pH-independent coating is desired, the coating is designed to achieve optimal release regardless of pH-changes in the environmental fluid, e.g., the GI tract. When the coating is formulated to release a compound according to this disclosure in the intestines (especially the upper small intestines), the coating is often called an “enteric coating”. A pH-dependent coating may include, but is not limited to, acrylic acid polymers and copolymers, for example polymers formed from acrylic acid, methacrylic acid, methyl acrylate, ammonio methylacrylate, ethyl acrylate, methyl methacrylate and/or ethyl methacrylate (e.g., Eudragit™); cellulosic polymers such as hydroxypropyl cellulose, hydroxyethyl cellulose, hydroxypropyl methyl cellulose, methyl cellulose, ethyl cellulose, cellulose acetate, cellulose acetate phthalate (CAP), cellulose acetate trimellitate, hydroxypropylmethyl cellulose phthalate, hydroxypropylmethyl cellulose succinate and carboxymethylcellulose sodium; shellac (purified lac); vinyl polymers and copolymers such as polyvinyl pyrrolidone, polyvinyl acetate, polyvinylacetate phthalate (PVAP), vinylacetate crotonic acid copolymer, and ethylene-vinyl acetate copolymers; zein; and salts and combinations thereof. Oral tablets and capsules typically have coatings.

### Definitions

Unless defined otherwise, all technical and scientific terms used herein have the meaning commonly understood by a person skilled in the art to which present disclosure belongs. The following references provide one of skill with a general definition of many of the terms used in present disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above-described cells and methods without departing from the scope of the present disclosure, it is intended that all matter contained in the above description and in the examples given below, shall be interpreted as illustrative and not in a limiting sense.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or items and those that do not materially affect the essential characteristics of the present disclosure.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the terms “disease”, “disorder” or “dysfunction” are used interchangeably in the present disclosure. They refer to any condition, disorder or disease manifested as one or more physiological, physical and/or psychological symptoms or dysfunctions for which treatment is desirable, and includes previously and newly identified diseases, disorders or dysfunctions on any organs, tissues or biological activities. As used herein, the term “medical use” is any use or means related to restore, remedy, or preserve health or well-being of a subject.

As used herein, the term “subject” means that preferably the subject is a mammal, such as a human, but can also be an animal, e.g., domestic animals (e.g., dogs, cats and the like), farm animals (e.g., cows, sheep, pigs, horses and the like) and laboratory animals (e.g., cynomolgus monkey, rats, mice, guinea pigs and the like).

As used herein, the administration of an agent or drug to a subject or patient includes self-administration and the administration by another. It is also to be appreciated that the various modes of treatment or prevention of medical conditions as described are intended to mean “substantial”, which includes total but also less than total treatment or prevention, and wherein some biologically or medically relevant result is achieved.

The publications discussed above are provided solely for their disclosure before the filing date of the present application. Nothing herein is to be construed as an admission that the present disclosure is not entitled to antedate such disclosure by virtue of prior disclosure.

### EXAMPLES

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Overview of Study Design

Until the discovery of insulin, Type 1 diabetes (T1D) was universally fatal. Insulin therapy has saved countless lives and the technology of delivering treatment continues to improve. But today, over a century later, insulin is still the only existing therapy for T1D—and it has significant limitations.

Insulin treatment does not truly normalize blood sugar control and often results in blood sugar volatility, a potentially dangerous condition. Other common side effects include hypoglycemia (below-normal blood sugar, which can be life-threatening) and weight gain. In addition, many people with T1D don't have access to the latest therapeutic devices. The most advanced insulin therapy technology—an insulin infusion pump with integrated continuous glucose monitoring (CGM), which allows for the most precise control—is currently available to only a small minority of T1D patients.

In summary, current treatments for T1D are centered on exogenous insulin administration, which is used to replace endogenous insulin produced and secreted by pancreatic islet beta cells that are destroyed via autoimmune mechanisms in T1D. Differences among these insulin-based therapies include the insulin formulation (which affects its duration of action) or the method of insulin delivery (e.g., direct subcutaneous injection, automated subcutaneous infusion pump). There are two major shortcomings with all these insulin-based therapies: 1) the risk of insulin-induced hypoglycemia, which can be fatal and 2) weight gain with long-term use of insulin. Finally, insulin-based treatment may be associated with frequent fluctuations of blood glucose levels throughout the day requiring frequent or continuous blood glucose monitoring. The present disclosure presents a novel treatment of T1D with brain delivery of a leptin active either through local delivery or systemic delivery. FIGS. 1A-1B illustrate the path of a local intrathecal delivery. As used herein, leptin active is a leptin compound, or a modified leptin compound. The leptin compound comprises a leptin, a leptin isomer, a leptin mimetic, a leptin analog, a leptin agonist, a leptin derivative, an anti-leptin receptor antibody, or any combination thereof. The modified leptin compound is a conjugate of the leptin compound with a Blood-Brain-Barrier (BBB) penetrating moiety. The BBB penetrating moiety comprises at least one fragment selected from the group consisting of a transferrin receptor, an insulin receptor, an insulin-like growth factor receptor, a low density lipoprotein receptor, a low density lipoprotein receptor-related protein 8, a low density lipoprotein receptor-related protein 1, a heparin-binding epidermal growth factor-like growth factor, an antibody thereof, or any combination thereof. Such intrathecal leptin-based treatment of hyperglycemia in T1D has several advantages: 1) results in more stable blood glucose control than achieved with insulin (i.e., maintenance within the normal range with less fluctuations), 2) does not carry the risk of hypoglycemia, and 3) does not result in weight gain. Furthermore, intrathecal leptin-based treatment would not require regular blood glucose monitoring as is currently required with insulin therapy.

The present disclosure relates to central nervous system administration of the leptin active (such as metreleptin or other leptin analog/isoform designed specifically for intrathecal administration). One embodiment comprises placing an intrathecal catheter in the high cervical spine, close to the craniocervical junction. This placement allows for targeted delivery of leptin to the brain and thereupon to treat hyperglycemia in people with T1D. The study design encompasses various aspects. One aspect relates to novel formulations or compositions to improve bioavailability, uptake and/or accumulation of the leptin actives in the brain. Such formulations or compositions may improve the delivery and/or release profile of the leptin active, such as enhanced or improved permeation or penetration of BBB, extended half-life, improved stability, and/or reduced toxicity of the leptin active. Different formulations or compositions are to be developed, such that these formulations may have dosing schedules that range from a day to weeks between doses. Another aspect relates to delivery devices capable of enhancing delivery of leptin analog/isoform to the cerebrospinal fluid targeting the brain by using implanted subcutaneous or transcutaneous reservoirs or port devices. These reservoirs or devices are connected at the proximal end of the intrathecal catheter, whereby leptin is administered percutaneously or transcutaneously. Such set up aids in the delivery of leptin to the cerebrospinal fluid via the intrathecal catheter. These devices include reservoirs that store the leptin drug subcutaneously for periods up to several days prior to administration through the catheter. Alternative, the delivery can be achieved by implanted subcutaneous automated infusion pumps (leptin drug would fill the pump, which would then deliver the drug continuously or in a scheduled manner, such as a daily bolus dose, over a period of time with percutaneous refills of the pump required when leptin levels in the pump are low). Another option is to use intrathecal catheter with distal aperture design that improves rostral flow of drug in the cerebrospinal fluid for targeted brain delivery.

A study involves placing an intrathecal catheter with its distal tip in the high cervical spine to deliver leptin through the cerebrospinal fluid to target the brain. For example, Ascenda catheter made by Medtronic can be used for this study, although other commercially available intrathecal catheters may also be suitable. It is also possible to design intrathecal catheter or devices specific for this use. Metreleptin is used as the model drug. Metreleptin is a recombinant analog of the human hormone leptin, it is the active ingredient of drug product Myalept® marketed by Amryt Pharmaceuticals, which is approved by FDA for lipodystrophy. Studies on metreleptin start with preclinical safety testing of intrathecal metreleptin in animals, such as pigs, followed by clinical trials of intrathecal metreleptin in patients with Type 1 Diabetes. Next is to develop a different leptin analog or formulation that is specifically designed for intrathecal use and for the specific clinical indication of Type 1 Diabetes.

As for delivery device, one way is to use an established or commercialized implantable intrathecal drug delivery system with subcutaneous port access, such as those shown in FIGS. 2A-2B. Other approaches are to be developed, including systemic and/or intrathecal administration of other chemically modified forms of leptin, leptin analogs or leptin isoforms, as well as monoclonal antibodies and small molecule ligands targeting the leptin receptor. These therapeutic agents will bind to the leptin receptor with greater affinity than native leptin, therefore having a much longer duration of action, and/or have heightened permeability across the blood-brain barrier. These features facilitate parenterally delivery, such as delivery by subcutaneous route, by intramuscular route, or through intranasal route.

The target population in general is the T1D population. This novel treatment particularly benefits the T1D sub-population, wherein patients have difficulty with insulin treatment due to either frequent hypoglycemia episodes or who have significant weight gain—the two major drawbacks of insulin therapy. T1D patients with hypoglycemia associated autonomic failure or obesity may therefore be important target populations. There are at least two brain areas that the therapeutic agent can target simultaneously in order to achieve their anti-diabetic effect in T1D—these areas include but are not limited to, the arcuate nucleus, the ventromedial nucleus of the hypothalamus, or the nucleus of the solitary tract of the hindbrain.

### Example 2. Targeting Brain to Treat Type 1 Diabetes—Alternatives to Insulin Therapy

Until the discovery of insulin, T1D was universally fatal. Insulin therapy has saved countless lives and the technology of delivering treatment continues to improve. But today, over a century later, insulin is still the only existing therapy for T1D—and it has significant limitations. Insulin treatment does not truly normalize blood sugar control and often results in blood sugar volatility, a potentially dangerous condition. Other common side effects include hypoglycemia (below-normal blood sugar, which can be life-threatening) and weight gain. In addition, many people with T1D don't have access to the latest therapeutic devices. The most advanced insulin therapy technology, which is an insulin infusion pump with integrated continuous glucose monitoring (CGM), which allows for the most precise control, is currently available to only a small minority of T1D patients, leaving an urgent need to find alternatives to insulin therapy for T1D. The examples described herein focus on leptin.

The brain demonstrates a surprising ability to control blood sugar levels, even without insulin. The hormone leptin, which is generated by fat cells, is implicated. Leptin controls appetite and body weight, but it is also essential for blood sugar control. Because insulin deficiency causes leptin deficiency, patients with poorly controlled T1D have low amounts of both insulin and leptin. Studies of lipodystrophy (a rare disease that involves selective loss of fat tissue) support the hypothesis that leptin deficiency can cause diabetes. People with this disease have a leptin deficiency. They also have a severe form of diabetes that cannot be controlled effectively with insulin, but it can be completely reversed with leptin.

Since the brain is the main target organ for leptin action, the next question was whether leptin's anti-diabetic action is controlled by the brain. To test this idea, leptin was infused directly into the brain of animal models of T1D. It was found not only did central leptin administration fully normalize the blood sugar level, but it did also so with far less blood sugar variability and weight gain compared to insulin treatment. Unlike insulin, which simply lowers blood sugar from its starting value, leptin actually normalizes the control of blood sugar, eliminating the risk of hypoglycemia.

Studies have shown that leptin infusion directly to the brain, at doses much lower than those required with injection into the bloodstream, produce exceptionally reliable blood sugar control in models of T1D-without the need for insulin.

### Intrathecal Treatments of T1D

These promising discoveries show the potential of leptin as an alternative to insulin. Metreleptin is currently FDA-approved for use by subcutaneous injection to treat diabetes associated with lipodystrophy, but it has yet to be approved for administration by the intrathecal route. The series of studies presented herein are to test and treat T1D patients with intrathecal (into the cerebrospinal fluid) leptin administration. The first step is to conduct a series of animal studies to evaluate the safety of intrathecally delivered metreleptin. Specifically, a series of Good Lab Practices (GLP)-compliant studies were designed and performed to assess the safety of intrathecal metreleptin in two animal species-Sprague Dawley rats and Yorkshire Farm pigs. Pilot experiments were done which has validated this approach, so formal approval-seeking studies are ready to begin. Then, human studies will follow. This is a first-in-human trial. Up to 10 patients with T1D are to be enrolled for a 14-day inpatient study. Participants will receive leptin infusion via a catheter inserted into the fluid surrounding the spinal cord, with close monitoring of their blood sugar and other metabolic lab values. Because implantable pumps and reservoirs are already approved for intrathecal drug delivery by the Food and Drug Administration (FDA), a positive result from this feasibility study would immediately lead to larger outpatient studies. After this proof-of-concept study demonstrates that brain-directed leptin therapy can normalize blood sugar without insulin, the result will be a fundamentally novel approach to T1D treatment. The ability to target the brain to normalize blood sugar in T1D patients has an unprecedented impact on diabetes that goes far beyond one new treatment. Establishing the brain's ability to control blood glucose without the need for insulin would open a new chapter in the history of diabetes. Therefore, intrathecal leptin treatment for T1D is a treatment with the potential to move beyond insulin.

### Example 3. Brain Directed Leptin Treatment for Type 1 Diabetes: Animal Studies

Although insulin therapy has been indisputably lifesaving for those diagnosed with T1D, it does not reliably ameliorate hyperglycemia and is associated with serious adverse effects. In the century since its discovery, technological advances have provided improved methods for how and when to deliver insulin (or its analogues), but fundamental shortcomings of insulin therapy remain. Exogenous insulin cannot meet the high insulin requirements of its upstream targets, the hepatocytes and pancreatic islet alpha cells, without exceeding the low insulin requirements of its downstream targets, the adipocytes and skeletal muscle. This discrepancy in insulin requirement among tissues contributes to “euglycemic volatility”, the frequently dangerous lability in blood glucose (BG) levels that typifies T1D. Both hypoglycemia, which can be life-threatening, and weight gain are side effects of insulin therapy, with tighter BG control associated with greater hypoglycemia risk and greater weight gain. Furthermore, the current gold standard insulin therapy—delivered via an insulin infusion pump with integrated continuous glucose monitoring (CGM) enabling the most precise BG control—does not obviate these risks and is only available to an exceedingly small minority of T1D patients, usually receiving care at an academic endocrinology clinic. The vast majority of T1D patients in the U.S. and worldwide do not have access to insulin pumps or CGM, do not meet targets for glycemic control, and thus are at risk for diabetes complications, including vision loss, kidney failure, neuropathy, limb amputation, and cardiovascular disease. These shortcomings highlight the distinction between treatments that re-set the biologically defended level of glycemia in the normal range and those, like insulin, that simply lower the blood glucose level. Recent preclinical studies, investigating the hormone leptin and its anti-diabetic efficacy when delivered directly to the brain, have established the feasibility of achieving the former goal as an alternative to insulin therapy for T1D.

Studies presented here aimed at translating the remarkable anti-diabetic effect of brain directed leptin treatment from animal models of T1D to people living with the disease. These studies represented the direct translation of a basic research finding, which was replicated in multiple independent labs, into a clinical application in human patients with T1D. Should this proof-of-concept study demonstrate that in T1D patients, brain-directed therapies can restore the defended level of glycemia to normal without insulin, the outcome would open entirely new therapeutic approaches for these patients. The availability of FDA-approved implantable pumps and reservoirs that allow brain targeted intrathecal drug delivery, currently used for intractable pain and spasticity, means that positive results from this feasibility study are expected to lead immediately to larger, chronic outpatient studies and therapeutic translation. Not since the discovery of insulin has preclinically-proven therapy for T1D held so much promise to revolutionize diabetes treatment.

### Specific Aims

Leptin is an adipocyte hormone generated in proportion to body fat mass primarily known for its role in regulating body fat stores, but mounting evidence over the past decade has shown that leptin is also essential for glucose homeostasis. Early insights from both rodent models and T1D patients revealed that T1D is characterized not only by a severe deficiency of insulin, but also leptin. Additional clues came from rodent models and patients with congenital or acquired lipodystrophy—characterized by selective loss of adipose tissue and leptin deficiency—with severe insulin-resistant diabetes that is reversed upon leptin administration (metreleptin is a drug formulation of leptin that is currently FDA-approved for subcutaneous injection in lipodystrophic diabetes). Initial studies demonstrated that leptin, administered systemically at supraphysiological doses, is capable of normalizing glycemia in multiple rodent models of T1D in the absence of insulin. Compared directly with insulin therapy in these models, peripheral leptin administration was equally effective at improving glycemia but with less blood glucose variability and virtually no hypoglycemia risk, and with beneficial effects on lipid metabolism—in contrast to insulin, leptin suppressed lipogenesis and had beneficial effects on body weight. Based on leptin's known primary site of action in the brain, multiple independent labs then showed that intracerebroventricular (ICV) infusion of leptin, at doses much lower than those required systemically (and that produce no detectable change of plasma leptin levels), produced even more robust and reliable normalization of glycemia. Unlike insulin's dose-response effect to simply lower blood glucose, leptin action in the brain appears to restore glycemia to normal without inducing hypoglycemia, and without the need for insulin. Remarkably, ICV leptin treatment restored glucose tolerance to near normal, despite ongoing, total insulin deficiency and the failure to mount a detectable insulin response to a glucose load. This highlights the existence of insulin-independent mechanisms for rapid glucose lowering that are activated by leptin action in the brain. Biochemical and tracer dilution studies suggested this involves potent suppression of hepatic glucose production and an increase in glucose uptake in skeletal muscle, the heart, and brown adipose tissue (likely mediated via autonomic nervous system innervation of these tissues). However, despite its remarkable capability to normalize glycemia, brain directed leptin administration has not been translated into a therapeutic strategy for human T1D.

Based on the accumulated preclinical data, a recent study attempting translation—using subcutaneous administration of high dose metreleptin (˜3-5 mg/day) in T1D patients—demonstrated metreleptin was safe in this population and produced modest reductions of body weight and insulin dose, but the study fell short of demonstrating its primary outcome of improved glycemia. This shortfall may be attributed to key discrepancies between this study and the conclusive preclinical data. In animal models, ICV leptin administration produced far more robust and reproducible normalization of glycemia than could be achieved by peripheral administration. Non-human primate studies illustrate this distinction: ICV injection of leptin (1 mcg/kg) safely produced a 40-50% reduction of food intake while triple this dose given peripherally had no effect. Peripheral hyperleptinemia may be counterproductive to metabolic homeostasis, raising the possibility that with systemic administration, the leptin dose required to achieve its beneficial action in the brain may be opposed by deleterious effects of an excess of leptin in plasma. These observations establish that in preclinical models, leptin administered directly to the brain is capable of normalizing glycemia far more effectively than systemically administered leptin, but this has yet to be studied in human T1D.

Using neurosurgical tools and techniques that can faithfully replicate the preclinical approaches outlined above, the present disclosure is now poised to make this clinical translation with the following aims: Aim 1: Demonstrate the clinical safety and toxicity profile of intrathecal metreleptin (i.e., direct cerebrospinal fluid delivery of the drug formulation of leptin) in two animal models. Aim 2: Demonstrate the safety and efficacy of intrathecal metreleptin administration in an inpatient study of human subjects with T1D.

### Research Strategy

### Aim 1: Demonstrating the Clinical Safety and Toxicity Profile of Intrathecal Metreleptin (i.e., Direct Cerebrospinal Fluid Delivery of the Drug Formulation of Leptin) in Two Animal Models.

### Rationale

Metreleptin is approved by the United States Food and Drug Administration (FDA) for use by subcutaneous injection to treat diabetes associated with congenital or acquired lipodystrophy. But there are insufficient data on the clinical safety and pharmacological toxicity of the drug administered by the intrathecal route to justify FDA approval. Based on preclinical work demonstrating that the intrathecal route of leptin administration provides the most robust and reliable normalization of blood glucose in T1D, studies are to be performed to assess necessary safety and toxicity endpoints for intrathecally-administered metreleptin. A series of Good Lab Practices (GLP)-compliant studies were designed to assess the safety and toxicity profile of intrathecal metreleptin in two animal species-Sprague Dawley rats and Yorkshire Farm pigs per FDA standards.

### Animal Study Approach

GLP-compliant animal studies in Sprague-Dawley (SD) rats and Yorkshire Farm (YF) pigs were designed to assess the safety of intrathecal metreleptin administration at escalating doses. Tested were groups of animals received doses close to the planned clinical dosing regimen, and groups received doses far exceeding the maximum dose to be administered in humans. Intrathecal catheter placement in pigs was proven to be feasible by the pilot experiments. These results confirmed that this approach closely replicates the procedure in humans and precisely recapitulates the drug administration strategy planned in humans.

Intrathecal position of the catheter is confirmed via fluoroscopy with injection of intrathecal contrast dye both at the time of initial catheter placement and at the end of the study prior to euthanasia for tissue harvest. Due to anatomic size limitations, it is not feasible to place an indwelling intrathecal catheter in rats for a period of 11 days, which matches the duration of treatment in the planned clinical human study. Rats will therefore be given metreleptin via an implanted intracerebroventricular cannula, which will allow assessment of clinical and histological neurotoxicity with chronic, daily, direct metreleptin exposure over 11 days (FIGS. 3A-3B).

The parallel animal studies include 24 pigs and 60 rats, divided into 4 dose escalation groups (saline, low [5 mcg/kg/day], medium [10 mcg/kg/day], and high dose [100 mcg/kg/day]) receiving daily intrathecal metreleptin for 11 days, with behavioral clinical monitoring followed by post-mortem histopathology studies to confirm absence of toxicity (FIG. 4). Metreleptin is to be administered as a single daily bolus injection either ICV (rats) or intrathecal (IT; pigs), matching the dosing frequency of planned human study. Dose determinations were based on existing literature of intrathecal leptin administration across species. Each group includes subsets of animals that are used for either acute (terminated 24 h after last dose) or chronic (terminated 72 h after last dose) studies of gross and microscopic histopathology. Tables 1A-1B provide study details on pigs and rats. Table 2 provides dosing references.

TABLE 1A
Pig Study (via intrathecal [IT] catheter)

| | | Low | Medium | High |
|---|---|---|---|---|
| Treatment | Saline | (5 mcg/kg/d) | (10 mcg/kg/d) | (100 mcg/kg/d) |
| N | 4 acute | 4 acute | 4 acute | 4 acute |
| | 2 chronic | 2 chronic | 2 chronic | 2 chronic |
| Assessments | Neuro/Health | Neuro/Health | Neuro/Health | Neuro/Health |
| | Battery | Battery | Battery | Battery |
| | Histopathology | Histopathology | Histopathology | Histopathology |
| | (Gross + Micro) | (Collect/Archive) | (Collect/Archive) | (Gross + Micro) |
| | CSF | Plasma PK | Plasma PK | CSF |
| | analysis/culture | Day 1 & 11 | Day 1 & 11 | analysis/culture |
| | | (Freeze plasma) | (Freeze plasma) | Plasma PK |
| | | | | Day 1 & 11 |

TABLE 1B
Rat Study (via intracerebroventricular [ICV] cannula)

| | | Low | Medium | High |
|---|---|---|---|---|
| Treatment | Saline | (5 mcg/kg/d) | (10 mcg/kg/d) | (100 mcg/kg/d) |
| N | 10 acute | 10 acute | 10 acute | 10 acute |
| | 5 chronic | 5 chronic | 5 chronic | 5 chronic |
| Assessments | Neuro/Health | Neuro/Health | Neuro/Health | Neuro/Health |
| | Battery | Battery | Battery | Battery |
| | Histopathology | Histopathology | Histopathology | Histopathology |
| | (Gross + Micro) | (Collect/Archive) | (Collect/Archive) | (Gross + Micro) |
| | | Plasma PK | Plasma PK | Plasma PK |
| | | Day 1 & 11 | Day 1 & 11 | Day 1 & 11 |
| | | (Freeze plasma) | (Freeze plasma) | |

TABLE 2
Intrathecal leptin dosing based on average species weight and CSF volume

| | | | Leptin | | Species | | Leptin dose | | | | Leptin dose |
|---|---|---|---|---|---|---|---|---|---|---|---|
| | | | dose | | average | | (weight | | Species | | (CSF volume |
| Study | Species | Route | (absolute) | | weight | | based) | | CSF volume | | based) |
| Hidaka 2002¹³ | Rat | ICV-pump | 3 | mcg/day | 0.3 | kg | 10 | mcg/kg/day | 0.156 | ml | 19 mcg/ml |
| | | infusion | | | | | | | | | CSF/day |
| Lin 2002¹⁴ | Rat | ICV-bolus | 10 | mcg/day | 0.3 | kg | 33 | mcg/kg/day | 0.156 | ml | 64 mcg/ml |
| | | dose | | | | | | | | | CSF/day |
| Fujikawa 2010⁴ | Mouse | ICV-pump | 1.2 | mcg/day | 0.03 | kg | 40 | mcg/kg/day | 0.036 | ml | 33 mcg/ml |
| | | infusion | | | | | | | | | CSF/day |
| German 2011¹⁵ | Rat | ICV-pump | 1 | mcg/day | 0.3 | kg | 3.33 | mcg/kg/day | 0.156 | ml | 6.4 mcg/ml |
| | | infusion | | | | | | | | | CSF/day |

| | | | | | | | | | | |
|---|---|---|---|---|---|---|---|---|---|---|
| Tang-Christensen | R. | ICV-bolus | — | 10-14 | kg | 1 | mcg/kg/day | 13 | ml | 1 mcg/ml |
| 1999³⁰ | Macaque | dose | | | | | | | | CSF/day |

The following safety/toxicity endpoints are to be assessed. First, clinical observations/overall health assessments are performed routinely over the 11-day period for rats and pigs, including assessment of activity, posture, respiration, hydration status, stereotypic behavior, bladder and bowel (stool) observations, surgery site observation, and overall body condition. Neurological assessments in pigs include the Porcine Neurological Motor Score (PNM), hind limb clearance, sensory function (withdrawal response), allodynia assessment, and anal sensation. A reduction of food intake with possible weight loss is an anticipated biological effect of metreleptin administration rather than an adverse effect, unless associated with other signs of illness. Secondly, post-mortem brain and cervical spinal cord histopathology to be performed at the conclusion of the 11 days to assess for histological neurotoxicity at acute and chronic timepoints. This analysis includes the FDA-recommended sections for brain toxicity studies, with staining for H&E and special stains for neurons, glia, and myelin. Thirdly, CSF culture, cytology, and protein are assessed on CSF samples obtained at the time of catheter placement (day 1) and at the time of euthanasia (day 12/14) to assess for catheter-related infection. Fourthly, plasma pharmacokinetics (PK) are assessed on days 1 and 11 following metreleptin administration to determine levels of metreleptin that crossover from CSF to plasma both after a single dose (day 1) and after multiple repeated daily doses (day 11). It is anticipated this crossover being negligible based on prior studies with leptin.

Details of the tests include the following. Pigs undergo percutaneous placement of an IT catheter with the catheter tip terminating at the cervical two level to match the intended location in the human study. Rats undergo ICV cannula placement into the lateral ventricle to enable daily cerebrospinal fluid dosing, supplanting chronic IT catheter placement that is not feasible in rodents. Bolus dosing performed on 11 consecutive days (daily IT in pigs or ICV in rats) to match human study dosing. Acute animals are sacrificed 24 h post-last dose and chronic at 72 h post-last dose. Metreleptin doses were selected as follows: the low dose (5 mcg/kg/d) is the approximate dose to be administered in humans; the medium dose (10 mcg/kg/d) is double that dose and may be administered in humans if the low dose is ineffective and not associated with any adverse effects; the high dose (100 mcg/kg/d) is 10× the highest dose planned for administration in humans, which is the recommended high dose to test for biologics based on FDA guidance. Metreleptin lyophilized cake (11.3 mg) are reconstituted in its sterile storage vial with 2.2 ml water for injection (WFI) to a concentration of 5 mg/ml, as intended for subcutaneous use. This concentration is used for the high dose group. For the low and medium dose groups, reconstituted metreleptin are further diluted with WFI. In pigs, metreleptin is administered via the IT catheter in a 1 ml volume followed by a 1 ml preservative-free saline flush to clear the catheter (catheter dead space=0.74 ml). In rats, metreleptin is administered via ICV cannula in a 10 ul volume. The bolus dose is administered over 1-2 minutes. The metreleptin concentration, volume to be administered (relative to species CSF volume), and rate of delivery matches or exceeds that intended for humans. All animals have jugular venous catheterization to enable repeated plasma collection for PK analysis before and after dose administration on Day 1 and Day 11. PK is performed by GLP-validated ELISA immunoassay. Histopathology includes gross examination of the brain and spinal cord and microscopic examination of 7 brain sections and 1 spinal cord section. Sections will be stained for H&E, Iba-1 (microglia), and GFAP (astrocytes). CSF analyses are performed in pigs, including glucose, protein, cell count and culture to confirm no evidence of infection, at time of catheter placement (day 0) and at time of euthanasia (day 12/14). Neuro/Health Battery includes Animals are monitored daily with assessment of activity, posture, respiration, hydration status, stereotypic behavior, bladder and bowel (stool) observations, surgery site observation, and overall body condition. Body weight is recorded weekly. Detailed neurological assessments are conducted on days 3, 8, 11, and 14. For pigs, this includes the Porcine Neurological Motor Score (PNM), hind limb clearance, sensory function (withdrawal response), allodynia assessment, and anal sensation. For rats, this includes a neurological motor evaluation with hind limb clearance, sensory function (withdrawal response), allodynia assessment, and anal sensation. A reduction of food intake with possible weight loss would be an anticipated biological effect of metreleptin administration rather than an adverse effect, unless associated with other signs of illness. Histopathology and PK assessments are performed in the high dose (and saline) group and the necessary specimens will be collected and stored for the low and medium dose groups. If toxicity is observed in the high dose group, then these analyses in the low and medium dose groups are also to be performed.

The first-in-human studies on intrathecal metreleptin treatment for T1D (Aim 2) is to be followed after the successful execution of these studies by confirming the absence of clinical and histological toxicity with intrathecal metreleptin administration at doses to be administered in planned human study (Aim 2).

### Example 4. Intrathecal Leptin Treatment for Type 1 Diabetes: Clinical Proof-of-Concept and Feasibility Study

Aim 2: Demonstrate the Safety and Efficacy of Intrathecal Metreleptin Administration in an Inpatient Study of Human Subjects with T1D.

### Rationale

Preclinical studies have conclusively shown that brain directed leptin treatment is capable of normalizing glycemia in multiple animal models of T1D without insulin. Leptin treatment (in the absence of insulin) compares favorably to insulin therapy itself, with equal efficacy at improving glycemia but with less blood glucose variability and virtually no hypoglycemia risk, and with beneficial effects on lipid metabolism. In animal models of T1D, leptin restores not only basal glucose levels to normal, but also restores glucose tolerance to near normal, despite ongoing, total insulin deficiency and the failure to mount a detectable insulin response to a glucose load. However, despite these profound anti-diabetic effects in animal models of T1D, intrathecal leptin has yet to be proven to be translatable into a therapy for human T1D. The inpatient study described herein is conducted to demonstrate the safety and efficacy of intrathecal metreleptin administration in human subjects with T1D.

### A. Clinical Study Synopsis

A proof-of-concept first-in-human safety and feasibility trial is conducted to test whether intrathecal leptin administration restores normoglycemia to patients with T1D, even in the absence of insulin therapy (FIG. 5). This inpatient study enrolls up to ten patients with C-pepT1De negative Type 1 Diabetes (T1D) currently managed with an insulin pump. On Day 1, patients are admitted to the hospital and a tunneled intrathecal catheter terminating in the cervical spine is placed. Patients receive lunch with a normal mealtime insulin bolus through their home insulin pump. Approximately 2-3 hours later, the pump is removed, and patients are monitored in the intensive care unit using continuous glucose monitoring (CGM) and point-of-care (POC) testing, with their outpatient insulin regimen converted to an intravenous insulin infusion, using their pump-derived mean insulin dose per day to determine their entry point on the Insulin Infusion Algorithm (FIG. 6). After one day of recovery following catheter placement, intrathecal metreleptin administration (5-10 mcg/kg/day) is initiated as a single daily dose via the tunneled catheter. Patients are provided a low carbohydrate diet (˜25-30 g) and sub-cutaneous insulin will be administered at mealtime only, at approximately ½ the normal outpatient dose. Based on preclinical data, intrathecal leptin-mediated glycemic control is expected within 4-6 days following initiation of treatment, which is marked by a reduction in intravenous insulin infusion requirements. Although appetite and food intake may decrease, no adverse effects are expected from the central leptin treatment. As leptin therapy ensues, the insulin infusion is further titrated, potentially to zero, with the goal of demonstrating insulin-independent normalization of glycemia via the central action of leptin. Once the insulin infusion rate drops to zero, pre-meal insulin is withheld, at the discretion of the clinical investigator. The inpatient study duration for each patient plans is approximately 14 days, at the conclusion of which the intrathecal catheter is removed at the bedside, a stitch placed at the puncture site, and the patient converted back to their outpatient insulin regimen. Safety data, including drug-related toxicity, treatment-emergent adverse events, and clinically significant lab abnormalities are collected and monitored by a Data and Safety Monitoring Board.

### B. Primary and Secondary Study Objectives and Endpoints

The following primary and secondary endpoints are to be monitored.

TABLE 3
Primary and Secondary Study Objectives and Endpoints

| | Objectives | Endpoints |
|---|---|---|
| Primary | 1. Determine whether daily | 1. Number of T1D patients off |
| Objectives/Endpoints | intrathecal leptin | insulin therapy (IV insulin |
| | administration is capable | infusion off for at least 6 |
| | of normalizing blood | hours) that achieve a fasting |
| | glucose levels in patients | BG 70-125 mg/dl and beta- |
| | with Type 1 diabetes | hydroxybutyrate <0.6 mM |
| | without insulin therapy, as | during the inpatient stay (up |
| | has been observed in | to 14 days). |
| | preclinical models. | |
| Secondary | 1. Examine the effects of | 1. Number of T1D patients off |
| Objectives/Endpoints | daily intrathecal leptin | insulin therapy achieving |
| | administration on the | CGM Time-In-Range (TIR) |
| | determinants of glucose | (70-180 mg/dl) >70% for at |
| | homeostasis in patients | least 3 days |
| | with Type 1 Diabetes. | 2. Number of T1D patients off |
| | | insulin therapy achieving |
| | | CGM Time-Above-Range, |
| | | TIHyper^{CGM} (>180 mg/dL) <25% |
| | | for at least 3 days |
| | | 3. Number of T1D patients off |
| | | insulin therapy achieving |
| | | CGM time-below-range, |
| | | TIHypo^{CGM} (<70 mg/dl) <4% |
| | | for at least 3 days. |
| | | 4. Number of T1D patients off |
| | | insulin therapy achieving |
| | | CGM time-below-range, |
| | | TIHypo^{CGM} (<54 mg/dl) 0% |
| | | for at least 3 days. |
| | | 5. Number of T1D patients off |
| | | insulin therapy (≥6 hours) |
| | | with normal oral glucose |
| | | tolerance test (OGTT) |
| | | (BG <140 mg/dL at 3 hrs.). |
| | | 6. Median percentage change in |
| | | a) food intake, b) insulin dose, |
| | | c) plasma counterregulatory |
| | | hormones (glucagon, cortisol, |
| | | epinephrine, norepinephrine), |
| | | d) lipids/triglycerides/free |
| | | fatty acids and e) plasma |
| | | leptin level, when comparing |
| | | values at baseline to last day |
| | | of leptin treatment. |
| | | 7. Longitudinal trend in |
| | | glycemia over time per |
| | | patient measured using CGM |
| | | from time of hospital |
| | | admission to Day 14. |
| | | 8. Longitudinal trend in appetite |
| | | over time per patient |
| | | measured before each meal |
| | | using appetite visual analog |
| | | scale from time of hospital |
| | | admission to Day 14. |
| | 2. Safety and tolerability | a. Drug-related toxicity |
| | | b. Treatment-emergent adverse |
| | | events |
| | | c. Clinically significant |
| | | laboratory abnormalities |
| | | [including ketosis (β- |
| | | hydroxybutyrate ≥0.6, 1.5, |
| | | and 3.0 mM and |
| | | hypoglycemia (glucose ≤70 |
| | | and ≤54 mg/dL)] |

### C. Study Population

Each participant must meet all the following inclusion criteria and none of the exclusion criteria to be eligible for study entry.

TABLE 4
Inclusion and Exclusion Criteria
Inclusion Criteria:

| | |
|---|---|
| 1. | Is male, age 18-40 years old. |
| 2. | Is currently a non-smoker with no current indication of drug or alcohol abuse (as |
| | determined by the investigator). |
| 3. | Has been diagnosed with T1D for at least 1 year. Diagnosis of T1D will be based on |
| | clinical criteria including: insulin-dependence within 6 months of the onset, history of |
| | prior episode of ketoacidosis, previous documentation of positive serum diabetes |
| | autoantibodies (GAD, IA-2, Zinc transporter) or low or undetectable serum C-pepT1De |
| | levels. |
| 4. | Has an HbA1c <7.0%. |
| 5. | Has a BMI 20-27 kg/m2. |
| 6. | Currently treated by subcutaneous insulin pump + continuous glucose monitor (CGM); |
| | automated insulin delivery is not required but allowed |
| 7. | Has clinical laboratory test values (clinical chemistry, LFT, hematology, coagulation |
| | panel and urinalysis) determined to be not clinically significant by the investigator at |
| | screening visit. |
| 18. | Has a physical examination and electrocardiogram (ECG) with no clinically significant |
| | abnormalities as determined by the investigator. |
| 9. | Ability to understand and the willingness to sign a written informed consent document |
| | (personally or by the legally authorized representative, if applicable). |
| 10. | Participant has voluntarily agreed to participate by giving written informed consent |
| | (personally or via legally authorized representative(s), and assent if applicable). |
| | Written informed consent for the protocol must be obtained prior to any screening |
| | procedures. If consent cannot be expressed in writing, it must be formally documented |
| | and witnessed, ideally via an independent trusted witness. |

Exclusion Criteria:

| | |
|---|---|
| 1. | Have diabetic complications [clinical neuropathy or evidence of autonomic dysfunction |
| | (retinopathy ok), nephropathy (e.g., eGFR <60 mL/min)]. |
| 2. | Currently taking systemic glucocorticoids or sex steroids (thyroid replacement |
| | acceptable but must be confirmed euthyroid). |
| 3. | Currently taking sympatholytic or sympathomimetic agents (e.g., beta-agonists for |
| | asthma). |
| 4. | Have a malignant neoplasm. |
| 5. | Has received any investigational drug within 30 days or within a period corresponding |
| | to five half-lives of that drug, whichever is greater, before screening. |
| 6. | INR >1.3 or known coagulopathy. |
| 7. | Any known hematological abnormality (or complete blood count lab value outside the |
| | normal range). |
| 8. | Unstable cardiovascular disease [untreated cardiac arrythmia, myocardial infarction in |
| | last year, NYHA Functional Class III or IV heart failure, active peripheral vascular |
| | disease]. |
| 9. | Uncontrolled hypertension (SBP >160 or DBP >100) at screening. |
| 10. | Any known hepatic abnormality (or AST/ALT >3x upper limit of normal), clinically |
| | significant respiratory disease, clinically significant gastrointestinal disease, any other |
| | currently unstable or moderate to severe medical illness, or any disruptive psychiatric |
| | disorder that in the opinion of the investigator may adversely affect participant in study. |
| 11. | Has a known hypersensitivity to any of the components of the study treatment (e.g., |
| | has a known hypersensitivity to E. coli derived proteins). |
| 12. | Has previously received treatment with recombinant leptin (metreleptin or Fc leptin). |
| 13. | A known recent SARS-CoV-2 positive RT-PCR or molecular test result from any |
| | available respiratory tract sample (e.g., nasal swab sample, sputum sample, throat |
| | swab sample, saliva sample) or other sample or; COVID-19 symptoms consistent with |
| | those defined by the US FDA. |

### D. Study and Participant Duration.

The study duration will be approximately 2 months for startup, up to 12 months for enrollment, and then approximately 2 months for data analysis after enrollment is complete. Each participant participates for up to ˜2.5 months, including a screening visit-28 days prior to Day 1 (hospital admission) through the safety follow-up call ˜30 days after hospital discharge.

### E. Study Location

The study takes place at multiple hospitals with qualified facilities and trained personnels.

### F. Study Intervention Description

Percutaneous intrathecal catheter placement (Ascenda catheter #8782, Medtronic, Inc.) occurs on the day of admission (Day 1) in the interventional radiology biplanar fluoroscopy suite. Under fluoroscopic guidance, a 16-gauge Tuohy needle is used to gain intrathecal access percutaneously entering at the lumbar 2-3 interspace (or below) after suitable administration of local anesthetic (such as 1% lidocaine/0.25% bupivacaine with 1:400,000 epinephrine). The intrathecal catheter is then advanced through the Tuohy needle under fluoroscopic guidance from the lumbar 2-3 level to the cervical 2 level. The catheter is secured at the lumbar skin puncture site using an anchoring sleeve and 3-0 nylon suture. The externalized portion of the catheter is further secured to the skin with adhesive tape and the catheter is capped until Day 2, when daily intrathecal metreleptin administration is initiated.

Patients are admitted to the intensive care unit after catheter placement and will remain there for the duration of the study. They may have two antecubital IV lines placed, one used for insulin IV infusion (and dextrose infusion if necessary), and the other used for blood draws for diagnostic tests.

MYALEPT (metreleptin for injection) is a recombinant human leptin analog for injection that binds to and activates the leptin receptor. Metreleptin (recombinant methionyl-human leptin) is produced in E. coli and differs from native human leptin by the addition of a methionine residue at its amino terminus. Metreleptin is a 147-amino acid, nonglycosylated, polypepT1 De with one disulfide bond between Cys-97 and Cys-147 and a molecular weight of approximately 16.15 kDa. MYALEPT is supplied as a sterile, white, solid, lyophilized cake containing 11.3 mg that is reconstituted with 2.2 mL of WFI to a final formulation of 5 mg/mL metreleptin for subcutaneous injection. For intrathecal injection, metreleptin will be further diluted with WFI to achieve a concentration such that the patient dose is between about 5.0 mcg/kg/day to about 10.0 mcg/kg/day when administered as a single daily dose in a 4 ml volume. The 4 ml volume of metreleptin in WFI is delivered over 1 minute as a bolus each day, with 1 ml preservative-free saline used to flush the catheter following the metreleptin bolus (total catheter dead-space=0.74 ml). The metreleptin daily dose starts at 5.0 mcg/kg/day. With approval from the study's Data Safety and Monitoring Board (DSMB), investigator may increase dose up to 10.0 mcg/kg/day on Day 7 or later if fasting blood glucose has not dropped below the threshold of 150 mg/dL (dose adjusted through an increase in bolus concentration with volume delivered to remain at 4 ml). The formulation comprises inactive ingredients, such as glutamic acid (1.47 mg/mL), glycine (20 mg/mL), polysorbate 20 (0.1 mg/mL), and sucrose (10 mg/mL), and pH at 4.2536.

Single daily Metreleptin dose administration is initiated via the intrathecal catheter on approximately Day 2 in the morning and discontinued on Day 12 in the morning once the 11th dose has been completed (11 total doses administered as a single daily dose on Day 2 through Day 12). On Day 12, after the last dose is administered, the intrathecal catheter is removed at bedside, a stitch placed at the skin puncture site, and the patient converted back to his outpatient insulin regimen and monitored overnight for stable normoglycemia prior to discharge on Day 13.

If three (3) participants meet the primary endpoint, the investigator has the option in subsequent participants of stopping daily metreleptin dose administration on Day 9 or Day 10 to confirm the glucose lowering was due to leptin. If the glucose levels do increase and reach a level of ≥300 mg/dL, the patient is to be converted back to their outpatient insulin regimen, the intrathecal catheter is to be removed at bedside, a stitch placed at the skin puncture site, and the patient is to be monitored overnight for stable normoglycemia prior to discharge. The patient has the Day 12 scheduled assessments completed on the day the leptin treatment is stopped (e.g., Day 9 or Day 10). The patient may be discharged the following day. If the participant's glucose levels do not increase to reach 300 mg/dL, the patient is to remain inpatient for monitoring until Day 12, when the outpatient insulin regimen is initiated and the patient monitored for stable normoglycemia overnight prior to discharge, completing safety assessments per the schedule of events shown in

TABLE 5
TABLE 5: Schedule of Events During the Trial

| | | | | | | | | | | | | | | | End | Safety |
|---|---|---|---|---|---|---|---|---|---|---|---|---|---|---|---|---|
| | | | | | | | | | | | | | | | of | Fol.- |
| | Screening | | | | | | | | | | | | | | [symbol] | up |
| | −28 | Day | Day | Day | Day | Day | Day | Day | Day | Day | Day | Day | Day | Day | ±3 | ±5 |
| Visit | to −1 | 1 | 2 | 3 | 4 | 5 | 6 | 7 | 8 | 9 | 10 | 11 | 12 | 13 | Days | Days |
| Informed | X | | | | | | | | | | | | | | | |
| Consent | | | | | | | | | | | | | | | | |
| Inclusion/ | X | | | | | | | | | | | | | | | |
| Exclusion | | | | | | | | | | | | | | | | |
| Demographics | X | | | | | | | | | | | | | | | |
| Medical | X | | | | | | | | | | | | | | | |
| History | | | | | | | | | | | | | | | | |
| Prior/ | X | X | X | X | X | X | X | X | X | X | X | X | X | X | X | |
| Concomitant | | | | | | | | | | | | | | | | |
| Medication | | | | | | | | | | | | | | | | |
| Chemistry | X¹ | | X¹ | | X¹ | | | | | | | | X¹ | | X¹ | |
| (BMP) | | | | | | | | | | | | | | | | |
| Liver Function | X¹ | | | | | | | | | | | | | | | |
| Test (LFT) | | | | | | | | | | | | | | | | |
| Hematology | X¹ | | X¹ | | X¹ | | | | | | | | X¹ | | X¹ | |
| Coagulation | X¹ | | | | | | | | | | | | | | | |
| (PT/INR, PTT) | | | | | | | | | | | | | | | | |
| Endocrine | | | X² | | X² | | X² | | X² | | X² | | X² | | | |
| Metabolism | | | | | | | | | | | | | | | | |
| Labs | | | | | | | | | | | | | | | | |
| % HbA1c | X | | | | | | | | | | | | | | | |
| Urinalysis | X¹ | | | | | | | | | | | | | | | |
| Continuous | | ------X------- | | | | | | | | | | | | | | |
| Glucose | | | | | | | | | | | | | | | | |
| Monitoring | | | | | | | | | | | | | | | | |
| (CGM) | | | | | | | | | | | | | | | | |
| Blood | | ------X^{3, 7}------- | | | | | | | | | | | | | | |
| Glucose | | | | | | | | | | | | | | | | |
| β- | | | ------X^{4, 7}------- | | | | | | | | | | | | | |
| hydroxybutyrate | | | | | | | | | | | | | | | | |
| Hospital | | ------X------- | | | | | | | | | | | | | | |
| Stay | | | | | | | | | | | | | | | | |
| Biomarker | X | | | | | | | | | | | | X | | | |
| (plasma and | | | | | | | | | | | | | | | | |
| serum) | | | | | | | | | | | | | | | | |
| Hospital | | | | | | | | | | | | | | X | | |
| Discharge | | | | | | | | | | | | | | | | |
| Intrathecal | | X | | | | | | | | | | | X | | | |
| Catheter | | | | | | | | | | | | | | | | |
| Placement/ | | | | | | | | | | | | | | | | |
| Removal | | | | | | | | | | | | | | | | |
| Insulin IV | | ------X------- | | | | | | | | | | | | | | |
| Infusion⁵ | | | | | | | | | | | | | | | | |
| Metreleptin | | | X⁶ | X⁶ | X⁶ | X⁶ | X⁶ | X⁶ | X⁶ | X⁶ | X⁶ | X⁶ | X⁶ | | | |
| Administration | | | | | | | | | | | | | | | | |
| Oral Glucose | | | | | ------X⁷------- | | | | | | | | | | | |
| Tolerance Test | | | | | | | | | | | | | | | | |
| (OGTT) | | | | | | | | | | | | | | | | |
| OGTT | | | | | ------X⁷------- | | | | | | | | | | | |
| Laboratory | | | | | | | | | | | | | | | | |
| Tests | | | | | | | | | | | | | | | | |
| Appetite | | X | X | X | X | X | X | X | X | X | X | X | X | X | | |
| VAS | | | | | | | | | | | | | | | | |
| score | | | | | | | | | | | | | | | | |
| Food Intake | | X | X | X | X | X | X | X | X | X | X | X | X | | | |
| Diary⁸ | | | | | | | | | | | | | | | | |
| Fluid | | | X | X | X | | | | | | | | | | | |
| Intake/ | | | | | | | | | | | | | | | | |
| Output | | | | | | | | | | | | | | | | |
| Diabetes | X | X | X | X | X | X | X | X | X | X | X | X | X | | | |
| Medication | | | | | | | | | | | | | | | | |
| Log | | | | | | | | | | | | | | | | |
| Physical | X | | | | | | | | | | | | X | | X | |
| Exam | | | | | | | | | | | | | | | | |
| Neurological | X | | | | | | | | | | | | X | | | |
| Exam | | | | | | | | | | | | | | | | |
| Height | X | | | | | | | | | | | | | | | |
| Weight | X | X | X | X | X | X | X | X | X | X | X | X | X | | X | |
| BMI | X | | | | | | | | | | | | X | | X | |
| EKG⁹ | X | | | | | | | | | | | | | | | |
| Vital Signs¹⁰ | X | | | | | | | | | | | | X | | X | |
| Oral | | ------X¹¹------- | | | | | | | | | | | | | | |
| temperature | | | | | | | | | | | | | | | | |
| Adverse | X | X | X | X | X | X | X | X | X | X | X | X | X | X | X | X¹⁴ |
| Event | | | | | | | | | | | | | | | | |
| Monitoring¹² | | | | | | | | | | | | | | | | |

| | | |
|---|---|---|
| Safety | X¹³ | X¹⁴ |
| Phone | | |
| Call | | |

¹Laboratory assessments completed at screening visit (basic chemistry, hematology, LFT, and urinalysis)); on Day 2, the labs will be collected prior to insulin IV dose reduction; on Day 4, prior to the first metreleptin dose administration; on Day 14, following the final metreleptin dose administration; and at the End of Study visit. Blood collected as a morning fasting level.
²Endocrine metabolism labs to include free fatty acid, lipid panel, glucagon, insulin, C-pepT1De, cortisol, leptin, epinephrine, and norepinephrine. On Day 2, the labs will be collected prior to insulin IV dose reduction; on Day 4, prior to the first metreleptin dose administration; then every other day until Day 14, following the final metreleptin dose administration.
³Fasting glucose blood draw will be collected in the morning daily starting on Day 2 and sent to the clinical lab. Fingerstick point-of-care (POC) blood glucose testing will be done at the bedside hourly starting on Day 1 (first test just prior to beginning insulin IV infusion) until discharge.
⁴Fasting β-hydroxybutyrate blood draw will be collected in the morning daily starting on Day 2 and sent to the clinical lab. Fingerstick POC ketone testing will be done every 2 hours starting on Day 2 (first test just prior to insulin IV dose reduction) until discharge.
⁵Day 1: Convert to insulin IV infusion following the Insulin infusion algorithm guidelines (Appendix) approximately 2 hours after lunch; study CGM.
⁶Intrathecal metreleptin (5-10 mcg/kg/day) will be administered as a single daily dose via the tunneled catheter beginning on Day 2 AM through Day 12 AM..
⁷Oral glucose tolerance test (OGTT) will be completed on the first day when the insulin IV infusion has been off at least 6 hours and the fasting blood glucose is 70-125 mg/dl. Blood samples for testing will be collected at 0, 1, 2, and 3 hours. Testing will include Glucose, insulin, c-pepT1De, glucagon, GLP-1. POC testing of glucose and β-hydroxybutyrate will be completed every 30 min during OGTT. If the POC blood glucose is >450 mg/dl or the β-hydroxybutyrate is ≥0.6 mmol/l, the OGTT will be stopped, and the participant will receive an appropriate amount of insulin based Insulin infusion algorithm guide (Appendix).
⁸Food intake monitored (calorie/carbohydrate count). Patients will have low carbohydrate (~25-30 g/meal) menu selection. Sub-cutaneous insulin will be administered at approximately ½ the normal dose at mealtime until patients are no longer receiving IV insulin.
⁹EKG will be completed at screening visit and captured in the database. Continuous cardiac monitoring during the hospital stay will occur as part of routine ICU care. Any clinically significant abnormalities will be captured as an adverse event in the electronic database capture (EDC) system.
¹⁰Vital signs include heart rate, blood pressure, oral temperature, and pulse oximetry. Vital signs will be monitored continuously during the hospital stay as part of routine care; the assessments noted above will be collected in the EDC. Any clinically significant abnormalities will be captured as unscheduled assessments and as an adverse event in the EDC.
¹¹Oral temperature will be taken twice daily in the AM and PM. If a temperature is recorded that is ≥38° C., the temperature will be repeated approximately 1 hour later to confirm.
¹²Adverse Events will be captured following first administration of study drug. All serious adverse events will be recorded with start dates occurring any time after informed consent is obtained. AEs and SAEs will be captured until 30 days after the last day of study participation.
¹³Safety follow up call daily starting the day after discharge until the End of Study Visit. The End of Study visit will occur approximately 6 days after discharge.
¹⁴Safety follow up call approximately 30 days after discharge. Review for adverse events, including symptoms (i.e. fever) that may represent development of neutralizing antibodies. Patient may be asked to complete an in-clinic visit to be assessed if clinically indicated. [symbol]
indicates data missing or illegible when filed

### H. Efficacy Assessment

Efficacy assessment includes at least the following biomarkers: % HbA1c

The percentage hemoglobin A1c measures the fraction of hemoglobin in red blood cells that is glycosylated (i.e., covalently bound to glucose). This value estimates the average blood sugar over the previous 3 months. The range of values is 4.0% (better glucose control) to 14% (or higher; worse glucose control), with <6% being normal, 6-6.4% reflecting prediabetes, and >6.4% indicating diabetes. HbA1c percentage will be measured at screening only to determine eligibility.

### Blood Glucose

Fasting blood glucose measures the blood glucose level in the basal state (i.e., after a fast of at least 8 hours), not affected by recent food intake. The range of values can span from 50 mg/dL (or less; reflecting severe hypoglycemia, i.e., worse) to 70-99 mg/dL (reflecting normoglycemia, i.e., good) to 100-125 mg/dL (reflecting prediabetes level hyperglycemia, i.e., borderline between better and worse) to greater than 125 mg/dL (reflecting diabetes level hyperglycemia, i.e., worse). Fasting blood glucose will be measured at the screening and follow-up visits as part of the chemistry laboratory tests and in the morning during the inpatient stay. Additional blood glucose measurements will be completed via fingerstick point-of-care (POC) testing at other timepoints as noted in the schedule of events.

### β-Hydroxybutyrate

Fasting β-hydroxybutyrate (i.e., after a fast of at least 8 hours) will be collected in the morning during the inpatient stay. Additional β-hydroxybutyrate measurements will be completed via fingerstick POC testing at other timepoints as noted in the schedule of events.

### Continuous Glucose Monitoring (CGM)

CGM is continuous glucose monitoring data derived from continuous measures of interstitial fluid glucose taken at regular intervals throughout the day and night. The range of values can span from 50 mg/dL (or less; reflecting severe hypoglycemia) to over 350 mg/dL (reflecting hyperglycemia). This data may be analyzed to determine several consensus CGM metrics validated to accurately reflect blood glucose control over time: average glucose (AG), glucose management index (GMI), time in range (TIR), time in hyperglycemia (TIHyper), time in hypoglycemia (TIHypo), and glucose variability-coefficient of variation (GV-CV). The CGM device used to record glucose data for this study will be the same (Dexcom G6 Pro) for all patients, regardless of the personal CGM device used by the patient outside the study. Patients may be asked to provide the CGM data from their personal devices for up to 14 days prior to hospital admission.

### AG-CGM

AG is the average glucose measured using CGM over a defined period of time. The normal range for AG is 88-116 mg/dL in subjects without diabetes.

### GMI-CGM

GMI is the Glucose Management Index and is a value calculated from the mean CGM glucose with units (%) similar to the HgbA1c. It is intended to be a metric that estimates the HgbA1c while not directly linked to the laboratory HgbA1c value. The normal range for GMI is <6% in subjects without diabetes, similar to the range for HgbA1c.

### TIR-CGM

TIR is the time-in-range and is calculated as the percentage of CGM glucose values that fall within a desired range, most commonly 70-180 mg/dL by default. Values range from 0% (no CGM values in range, worst) to 100% (all CGM values in range, best).

### TIHyper-CGM

TIHyper is the time-in-hyperglycemia and is calculated as the percentage of CGM glucose values that are above a desired range, most commonly >180 mg/dL by default. Values range from 0% (no CGM values in hyperglycemia, best) to 100% (all CGM values in hyperglycemia, worst).

### TIHypo-CGM

TIHypo is the time-in-hypoglycemia and is calculated as the percentage of CGM glucose values that are below a desired range, most commonly <70 mg/dL by default. Values range from 0% (no CGM values in hypoglycemia, best) to 100% (all CGM values in hypoglycemia, worst).

### GV-CV-CGM

GV-CV is glucose variability measured as the coefficient of variation, which measures the amplitude of fluctuations of individual CGM glucose readings away from the mean CGM glucose. GV-CV<36% represents low glucose variability and a relatively stable glucose profile, while GV-CV>36% indicates high glucose variability and an unstable glucose profile.

### Oral Glucose Tolerance Test

Oral glucose tolerance test (OGTT) will be completed on the first day when the insulin IV infusion has been off at least 6 hours and the fasting blood glucose is 70-125 mg/dL. Blood samples for testing will be collected at 0, 1, 2, and 3 hours. Testing will include Glucose, insulin, c-pepT1De, glucagon, GLP-1. POC testing of glucose and β-hydroxybutyrate will be completed every 30 min. If the glucose is >450 mg/dL or the β-hydroxybutyrate is >0.6 mmol/l, the OGTT will be stopped, and the participant will receive an appropriate amount of insulin based on the insulin infusion algorithm guide (see Appendix).

### Meal Log

Patients and/or research staff will complete a meal log daily during hospitalization to record food, the percentage of meals eaten and the approximate caloric intake. During the in-patient stay, patients will be provided a low carbohydrate (˜25-30 g) menu selection.

### Appetite Visual Analog Scale Scoring

Patients will complete an Appetite Visual Analog Score (VAS) consisting of a line of 100 mm, the extremes anchored to “no hunger” (0 mm) and “hunger” (100 mm) prior to each meal per the Schedule of Events.

### Type 1 Diabetes Medication Use

Patient's typical outpatient insulin regimen will be documented, including mean insulin dose per day.

### Biomarker Collection

One sample will be collected for plasma (˜5 mL) and one sample for serum (˜5 mL) per the Schedule of Events. Samples will be frozen and stored for future research. Venipuncture may cause some pain, bleeding or bruising where the needle entered the participant's body. Some participants may have an intravenous (IV) catheter placed or a midline catheter for blood collection, as determined needed by the investigator. Placing an IV may cause some pain, and bleeding or bruising at the spot where the needle entered the participant's body. There may also be a risk of irritation of the vein (phlebitis), infection, blood clot, leakage, or infiltration and/or nerve or tendon injury during insertion. Care is taken to avoid these problems. The longer an IV catheter is left in place, the more common it is for redness or infection to develop. This information is included in the participant's informed consent and discussed with the participant as needed.

### I. Safety and Other Assessment

### Demographics

Demographic information (date of birth, gender, race, ethnicity) are recorded at Screening.

### Medical History

Relevant medical history, including history of current disease, other pertinent history, and information regarding underlying diseases are recorded at Screening.

### Hospital Admission

Hospital admission and discharge dates are recorded.

### Imaging

Fluoroscopic X-ray for catheter placement is conducted.

### Prior and Concomitant Medication

For this protocol, a prescription medication is defined as a medication that can be prescribed only by a properly authorized/licensed clinician. Medications to be reported in the Case Report Form (CRF) are concomitant prescription medications, over-the-counter medications, and supplements. All concomitant medication and concurrent therapies administered within 30 days of study entry are documented at Screening and during the study. Dose, route, unit frequency of administration, and indication for administration and dates of medication are captured.

### Physical Examination

Physical examinations include an examination as per standard of care and if available, are obtained through the review of existing data in the medical record. The exam may include a review of general appearance, skin, neck (including thyroid), eyes, ears, nose, throat, lungs, heart, abdomen, lymph nodes, extremities. The physical exam should be completed by appropriately licensed personnel as per institutional guidelines or as delegated by the Investigator.

### Vital Signs

Vital signs include body temperature, blood pressure, pulse, pulse oximetry and respiration rate measurements and if available, may be obtained through the review of existing data in the medical record. If not available, the assessment should be completed per the Schedule of Events. Blood pressure (systolic and diastolic) and pulse should be measured after the participant has been sitting for five minutes.

### Height and Weight

Height in centimeters (cm) and body weight (to the nearest 0.1 kilogram [kg] in indoor clothing, but without shoes) are measured. Height is measured only at screening. May be obtained through the review of existing data in the medical record. Body Mass Index (BMI) will be calculated.

### Electrocardiogram (ECG)

A standard 12-lead ECG is obtained as per the assessment schedule. All ECGs should be obtained after the participant has rested in a supine position for at least 5 minutes on a machine that automatically calculates heart rate and determines intervals for PR, QRS, QC and QTc.

### Laboratory Assessments

Blood and urine will be obtained and sent to the local laboratory for a CBC/hematology, blood chemistry, and pregnancy test shown in Table 6 and as noted in per the Schedule of Events.

TABLE 6
Laboratory Assessments

| Test Name | Test Category |
|---|---|
| Hematology | Hgb, platelets, white blood cells (WBC), red blood cells (RBC), |
| | differential (basophils, eosinophils, lymphocytes, monocytes, |
| | neutrophils [% or absolute]) |
| Blood Chemistry | BUN/Creatinine Ratio (calculated), Calcium, Carbon Dioxide, |
| (BMP) - Fasting | Chloride, Creatinine with GFR Estimated, Glucose, Potassium, |
| | Sodium, Urea Nitrogen (BUN) |
| Liver function test | ALT, AST |
| Blood glucose | Daily morning fasting level |
| Blood β- | Daily morning fasting level |
| hydroxybutyrate | |
| Endocrine | free fatty acid, lipid panel [Total cholesterol, triglycerides, high- |
| Metabolism Panel | density lipoprotein (HDL) and low-density lipoprotein (LDL)], |
| | glucagon, insulin, C-pepT1De, cortisol, leptin, epinephrine and |
| | norepinephrine |
| OGTT Blood | Glucose, insulin, c-pepT1De, glucagon, GLP-1 |
| Testing | |
| Urinalysis | Macroscopic panel (dipstick) (bilirubin, blood, glucose, ketones, |
| (screening only) | WBC, pH, protein, specific gravity) |
| | Reflexive Microscopic panel (RBC, WBC, casts) |
| Coagulation | International normalized ration (INR) and pro-thrombin time (PT) or |
| (screening only) | Quick Test; activated partial thromboplastin time (PTT) |

### Hospital Discharge

Hospital admission and discharge dates are obtained and recorded.

### Safety Phone Call

Telephone or video visits are completed every day starting the day after discharge until the EOS visit, including a remote review of adverse events.

### Adverse Events

### Definition of Adverse Events (AE)

Adverse event means any untoward medical occurrence associated with the use of an intervention in humans, whether or not considered intervention-related (21 CFR 312.32 (a)). Adverse Events (AEs) will be captured following first administration of study drug. Table 7 list some common AEs and non-AEs.

TABLE 7
Common Adverse Events (AEs) and Non-AEs

AEs include:
Any deleterious change from the participant's
baseline status, including an increase in the
severity or frequency of a pre-existing abnormality or disorder;
Concurrent illnesses;
Injury or accidents;
Subjective symptoms considered unfavorable by the reporter;
Clinically significant physical examination,
laboratory, imaging, or physiological
testing abnormalities (abnormalities requiring
treatment or a change in medication are AEs);
Overdose
AEs NOT include:
Laboratory or test abnormalities that are not
considered clinically significant.
Incidental findings on imaging that are not
considered clinically significant.
Conditions for which a procedure was planned
prior to signing the informed
consent (e.g., elective knee replacement)
Conditions present at baseline which have not worsened.
Hospitalization solely to complete procedures, or
for social reasons such as respite care.
Cosmetic procedures.
Abnormal vital sign values that are not considered
clinically significant; or do not require medical
intervention; or interruption/change to study treatment; or, not
accompanied by clinical symptoms.

### Definition of Serious Adverse Events (SAE)

An adverse event (AE) or suspected adverse reaction is considered “serious” if, in the view of either the investigator or sponsor, it results in any of the outcomes listed in Table 8. Table 8 also provides some examples of non-serious AEs and guidelines about severity of AEs.

TABLE 8
Serious vs Non-Serious Adverse Events and Severity Marking

Serious AEs
death,
a life-threatening adverse event,
inpatient hospitalization or prolongation
of existing hospitalization, a persistent
or significant incapacity or substantial
disruption of the ability to conduct normal
life functions, or a congenital anomaly/birth defect.
other important medical events that may not result in death,
be life-threatening, or require hospitalization
may be considered serious when, based upon
appropriate medical judgment, they may jeopardize the
participant and may require medical or surgical
intervention to prevent one of the outcomes listed
in this definition. Examples of such medical events
include allergic bronchospasm requiring intensive
treatment in an emergency room or at home, blood
dyscrasias or convulsions that do not result in
inpatient hospitalization, or the development
of drug dependency or drug abuse.
Non-Serious AEs
Routine treatment or monitoring of the
studied indication, not associated with
any deterioration in condition.
Elective or pre-planned treatment for a pre-
existing condition that is unrelated
to the indication under study and has not worsened
since signing the informed consent.
Social reasons and respite care in the absence of any deterioration in the
participant's general condition.
Severity of an AE:
1 = Mild AE: Awareness of symptom, but
easily tolerated; usually transient
requiring no special treatment; does not
interfere with usual status or activities
2 = Moderate AE: May be ameliorated by simple
therapeutic measures; may interfere with usual activities
3 = Severe AE: Incapacitating, inability to perform usual activities
4 = Life threatening consequences; urgent intervention indicated
5 = Fatal AE

### Relationship to Study Intervention

All adverse events (AEs) must have their relationship to study intervention assessed by the clinician who examines and evaluates the participant based on temporal relationship and his/her clinical judgment. The degree of certainty about causality are graded using the categories below. In a clinical trial, the study product must always be suspected.

Definitely Related—There is straightforward evidence to suggest a causal relationship, and other possible contributing factors can be ruled out. The clinical event, including an abnormal laboratory test result, occurs in a plausible time relationship to study intervention administration and cannot be explained by concurrent disease or other drugs or chemicals.

Probably Related—There is evidence to suggest a causal relationship, and the influence of other factors is unlikely. The clinical event, including an abnormal laboratory test result, occurs within a reasonable time after administration of the study intervention, is unlikely to be attributed to concurrent disease or other drugs or chemicals, and follows a clinically reasonable response on withdrawal.

Potentially Related—There is some evidence to suggest a causal relationship (e.g., the event occurred within a reasonable time after administration of the trial medication). However, other factors may have contributed to the event (e.g., the participant's clinical condition, other concomitant events). Although an AE may rate only as “possibly related” soon after discovery, it can be flagged as requiring more information and later be upgraded to “probably related” or “definitely related”, as appropriate.

Unlikely to be related—A clinical event, including an abnormal laboratory test result, whose temporal relationship to study intervention administration makes a causal relationship improbable (e.g., the event did not occur within a reasonable time after administration of the study intervention) and in which other drugs or chemicals or underlying disease provides plausible explanations (e.g., the participant's clinical condition, other concomitant treatments).

Not Related—The AE is completely independent of study intervention administration, and/or evidence exists that the event is definitely related to another etiology.

Definitely, probably and potentially related will be defined as a reasonable suspected causal relationship to the study intervention when determining if the event is an adverse drug reaction (ADR).

### Expectedness

The Safety Officer is responsible for determining whether an adverse event (AE) is expected or unexpected. An AE will be considered unexpected if the nature, severity, or frequency of the event is not consistent with the risk information previously described for the study intervention.

### Time Period and Frequency for Event Assessment and Follow-Up

The occurrence of an adverse event (AE) or serious adverse event (SAE) may come to the attention of study personnel during study visits and interviews of a study participant presenting for medical care, or upon review by a study monitor.

All AEs including local and systemic reactions will be captured on the appropriate case report form (CRF). Information to be collected includes event description, time of onset, clinician's assessment of severity, relationship to study product (assessed only by those with the training and authority to make a diagnosis), and time of resolution/stabilization of the event. All AEs occurring while on study must be documented appropriately regardless of relationship. All AEs will be followed to adequate resolution. Any medical condition that is present at the time that the participant is screened will be considered as baseline and not reported as an AE.

Changes in the severity of an AE will be documented to allow an assessment of the duration of the event at each level of severity to be performed. AEs characterized as intermittent require documentation of onset and duration of each episode. The Investigator records adverse events from the time of treatment until 7 days after the last day of study participation (for non-serious AEs). All serious adverse events will be recorded with start dates occurring any time after informed consent is obtained until 30 days (for SAEs) after the last day of study participation. At each study visit, the investigator will inquire about the occurrence of AE/SAEs since the last visit. Events will be followed for outcome information until resolution or stabilization.

The site investigator or designee records adverse events. Investigator or designee should use correct medical terminology/concepts when recording AEs on the Adverse Event CRF. Avoid colloquialisms and abbreviations. Only one AE term should be recorded in the event field on the Adverse Event CRF. For all AEs, a diagnosis (if known) should be recorded on the Adverse Event eCRF rather than individual signs and symptoms. However, if a constellation of signs and/or symptoms cannot be medically characterized as a single diagnosis or syndrome at the time of reporting, each individual event should be recorded on the Adverse Event CRF. If a diagnosis is subsequently established, all previously reported AEs based on signs and symptoms should be nullified and replaced by one AE report based on the single diagnosis, with a starting date that corresponds to the starting date of the first symptom of the eventual diagnosis.

### Reporting Serious Adverse Event

The site investigator or designee reports any serious adverse event (SAE), whether or not it is considered study intervention related, including those listed in the protocol or investigator brochure and must include an assessment of whether there is a reasonable possibility that the study intervention caused the event. Study endpoints that are serious adverse events (e.g., all-cause mortality) must be reported in accordance with the protocol unless there is evidence suggesting a causal relationship between the study intervention and the event (e.g., death from anaphylaxis). In that case, the Sponsor-Investigator must immediately report the event to the FDA. Information about all SAEs is collected and recorded on the Serious Adverse Event Report Form; all applicable sections of the form must be completed to provide a clinically thorough report. The investigator must assess and record the relationship of each SAE to each specific study treatment (if there is more than one study treatment), complete the SAE Report Form within 24 hours of determining the adverse event as serious.

The original copy of the SAE Report Form must be kept at the study site. All SAEs are to be followed until satisfactory resolution or until the site investigator deems the event to be chronic or the participant is stable. The Sponsor-Investigator or designee will be responsible for notifying the Food and Drug Administration (FDA) of any unexpected fatal or life-threatening suspected adverse reaction as soon as possible, but in no case later than 7 calendar days after initial awareness of the event. In addition, the Sponsor-Investigator or designee must notify FDA and all participating investigators in an Investigational New Drug (IND) safety report of potential serious risks, from clinical trials or any other source, as soon as possible, but in no case later than 15 calendar days after the Sponsor-Investigator determines that the information qualifies for reporting.

### J. Safety Considerations

### Risk/Benefit Assessment

### Known Potential Risks

For known potential risks associated with MYALEPT, check the MYALEPT Product Label for additional information. And check the Ascenda intrathecal catheter Product Label for additional information on risks associated with the catheter.

### Contraindications

MYALEPT is contraindicated in patients with general obesity and in patients with prior severe hypersensitivity reactions to metreleptin or to any of the product components. Known hypersensitivity reactions have included urticaria and generalized rash.

### Known Potential Benefits

There may be no potential benefits for the participants in this study as this is an inpatient feasibility study with a short, finite duration of study drug administration.

### Assessment of Potential Risks and Benefits

The serious risks associated with subcutaneous metreleptin administration noted in prior clinical studies include development of anti-metreleptin antibodies with neutralizing activity and development of T-cell lymphoma. The clinical implications of anti-metreleptin antibodies with neutralizing activity are not well characterized due to the small number of cases reported. Patients are monitored for 30 days following the hospital discharge for adverse events. If there is a clinical suspicion for the development of neutralizing autoantibodies, as indicated by serious or severe infection, then patients are tested for neutralizing antibodies. Patients that have a positive test (i.e., seroconvert) then continue to be tested with quarterly samples until their levels return to baseline. Patients that develop an infection are to be managed clinically per routine care for the infection. The likelihood of this risk is further mitigated in the present study due to the fact that (1) a lower dose of metreleptin is used (50-100 fold lower dose than typical subcutaneous injection dose), (2) delivering the drug intrathecally (in an immune-privileged environment), and (3) delivering the drug for a short, finite duration (reducing exposure for potential antibody development). T-cell lymphoma has been reported in a small number of patients receiving subcutaneous metreleptin treatment in the MYALEPT lipodystrophy program, however, lymphomas have also been reported in patients with acquired generalized lipodystrophy not treated with MYALEPT. A causal relationship has therefore not been established. In the present study, this potential risk is further mitigated by excluding any patients with any hematological abnormality as described in the inclusion/exclusion criteria.

Hypoglycemia is an additional risk in patients receiving subcutaneous metreleptin with concurrent insulin treatment and this risk is mitigated in the present study because the patients are to be closely monitored in the ICU with CGM, and with the ability to make rapid adjustments in insulin dose via insulin IV infusion. The risk profile of this study is also supported by a prior metreleptin intrathecal study performed as part of the Amgen Metreleptin Obesity Program. There was a single treatment emergent adverse event noted in that study of 5 patients, in which 1 patient developed meningitis related to multiple revision procedures required to replace the intrathecal catheter.

The potential risks associated with placement of an intrathecal catheter include infection such as meningitis and cerebrospinal fluid leak. To mitigate the risk of infection, patients are administered antibiotics to cover skin flora throughout the duration of the trial with the externalized catheter (cephalexin or sulfamethoxazole/trimethoprim depending on patient tolerance). This is a routine practice in the trial hospital and the externalized catheter trials are well tolerated, with no infections reported to date (details below), for patients with intractable chronic pain or cancer pain undergoing intrathecal catheter trials for intrathecal morphine administration. The cerebrospinal fluid leak risk is also a potential risk that have not been observed in pain patients undergoing these catheter trials, likely due to the short duration of the catheter placement. The procedure planned for the present clinical study—placing an externalized intrathecal catheter terminating at the cervical 2 level—is commonly performed by both pain anesthesiologists and neurosurgeons to treat patients with refractory neck and head pain and has been described in the literature with a very low risk profile.

At the trial institution, no catheter-related complications have been observed in patients undergoing inpatient trials with externalized intrathecal catheters terminating at the cervical 2 level (as planned for the current clinical study). Furthermore, no issues were observed with catheter integrity in any intrathecal catheter placement regardless of catheter tip location, whether for performing an intrathecal drug trial or for long-term intrathecal pump implant, in over 275 catheter procedures performed over 4 years. The externalized intrathecal catheter placement procedure has been performed successfully in 15 of 15 patients to date with no complications, with all catheters placed as described above at the C2 level to treat refractory head and neck pain with intrathecal morphine. In this cohort, the duration of the indwelling intrathecal catheter ranged from 5 to 22 days, with a mean of 9.4 days and with 6 of 15 patients having a duration of 10 or more days (patients were in the inpatient ICU setting as intended in the clinical study). Coincidentally, 2 of these 15 patients also had diabetes (Type 2), including the patient with the 22-day duration intrathecal catheter. No complications were observed with this approach in the 15 patients to date, including no infections or other catheter-related complications. Prophylactic antibiotics are prescribed for these patients due to an abundance of caution, despite the lack of prior evidence of infection that needed to be addressed.

With respect to the potential higher risk of infection/poor wound healing in T1D patients, there are several points to consider. The percutaneous procedure involves only 2 needle punctures of the skin (no incision) and is performed on T1D patients with excellent glycemic control (HbA1c<7.0%) achieved via insulin pump (inclusion criteria). Infection risk in patients with diabetes is tied primarily to poor long-term glycemic control and/or diabetes complications that impair immune responsiveness, which combined with reduced tissue perfusion, lowers resistance to infection and impairs wound healing. As such individuals are excluded from the present study, it is not believed that the trial protocol is associated with infection risk different from the very low risk described above. Furthermore, due to this very low risk, in the routine clinical practice of placing externalized trial intrathecal catheters for refractory pain syndromes, patients with diabetes are not excluded (as in the cohort of 15 patients above) nor are there specific thresholds for glycemia that must be met for eligibility. Nevertheless, specific criteria for stopping a subject are added related to any sign or symptom of infection, out of an abundance of caution.

Additional support for the safety of this procedure in patients with diabetes comes from a recent prospective, randomized clinical trial evaluating the efficacy of an implanted spinal cord stimulation system to treat painful diabetic neuropathy. This study revealed that stimulator implant procedures in patients with diabetes (inclusion criteria allowed for Type 1 and Type 2 diabetes with HbA1c<10.0%) were associated with no greater incidence of infection/wound complications (5.6%) than observed in prior spinal cord stimulator implant studies in the general population. This data is particularly relevant to the present clinical study because: 1) spinal cord stimulator electrode placement is performed percutaneously in a manner very similar to placement of an intrathecal catheter, except that the electrode is placed intraspinally in the epidural space while the catheter is placed in the intradural (intrathecal) space, and 2) prior to permanent placement of the electrode and implanted pulse generator, patients undergo a 7-day externalized trial (i.e., epidural electrode is placed with proximal end exiting the skin and connected to an external pulse generator) to evaluate efficacy of the stimulation. During the trial period, the patient is at home performing their usual activities with an epidurally-placed electrode lead exiting the skin. Notably, of the 104 patients that underwent the percutaneous trial electrode placement, there were no infections or wound complications reported during the trial period in this study. In addition to the substantial similarity between this procedure and the present clinical protocol, it is also noted that the neurostimulation trial included patients with more poorly controlled diabetes (higher HbA1c) and with advanced diabetes complications (including neuropathy) compared to the present study, and yet it was conducted without increased risk of infection. These findings lend strong additional support to the safety of the present study.

The patients in this study may not benefit directly, but this study has the potential to be of great benefit to the larger community of T1D patients if it is successful. Although insulin is a life-saving treatment for T1D patients, it also has significant drawbacks, including risk of fatal hypoglycemia and weight gain with chronic treatment (leading to other metabolic abnormalities). Central leptin administration, if successful in this study as in preclinical models of T1D, restores normoglycemia without the risk of hypoglycemia and with beneficial effects on weight and body fat. Positive results from this inpatient feasibility study would therefore immediately inform larger outpatient studies—with chronic leptin administration via available intrathecal catheter-subcutaneous port systems—aimed at therapeutic translation.

### Participant Stopping Criteria

Individual stopping criteria of special interest are listed in Table 7 below. The procedure for exiting a subject from the study will include resumption of the Phase 1 algorithm, removal of the intrathecal catheter and closure of the puncture site with a single stitch, and then transitioning back to the patient's home insulin regimen via insulin infusion pump and CGM.

TABLE 7
Individual Stopping Criteria

Inability to place catheter with the tip at the C2 level.
Inability to suppress ketones to <0.6 mM with phase
2 insulin gtt algorithm, even after moving up
(shifting right) in the algorithm for two
consecutive hours.
Any sign (fever, catheter exit site
skin erythema, tenderness, swelling)
or symptom (headache, photophobia,
meningismus, malaise) of catheter infection.
Any evidence of catheter malfunction or obstruction,
such as encountering resistance upon administration
of metreleptin or inability to aspirate
CSF via the catheter.

### Study Stopping Rules.

Study stopping rules are if there are more than two participants experience individual stopping criteria as described above. The options listed in Table 8 may occur as a result of any of the above.

TABLE 8
Options after Occurrence of Study Stopping Rules

Halt subject dosing or study enrollment until the
toxicity data can be further studied by the DSMB
Amend the protocol to address any of the below:
Evaluate additional subjects in a particular
dose cohort or in each dose cohort to make the
study more sensitive to characterizing adverse events;
Implementation of smaller dose increases between dose cohorts;
Exclusion of certain participants thought to be
more at-risk for a particular adverse event.
Stop the study.

### K. Data Management and Statistical Analysis

### Data Management

Data collection is the responsibility of the clinical trial staff at the site under the supervision of the site investigator. The investigator is responsible for ensuring the accuracy, completeness, legibility, and timeliness of the data reported. All source documents should be completed in a neat, legible manner to ensure accurate interpretation of data.

Hardcopies of the study visit worksheets will be provided for use as source document worksheets for recording data for each participant enrolled in the study. Data may also be available in the Electronic Health Record (EHR). Data recorded in the electronic case report form (eCRF) derived from source documents and the EHR should be consistent with the data recorded on the source documents and in the EHR.

Clinical data (including adverse events (AEs), concomitant medications, and expected adverse reactions data) and clinical laboratory data will be entered into REDCap, a 21 CFR Part 11-compliant electronic data capture system. The data system includes password protection and internal quality checks, such as automatic valid value and valid range alerts, to identify data that appear inconsistent, incomplete, or inaccurate. REDCap uses secure encryption technology to protect Internet data exchanges. All system modifications and data entries will be logged on a real-time audit trail. Clinical data will be entered directly from the source documents.

### Statistical Analysis

This is a feasibility study to examine whether basic research on the effects of central leptin action in T1D models can translate into a clinical application in human patients with T1D. This is an exploratory study, and no formal statistical hypothesis tests will be performed. The sample size was justified based on previous feasibility studies testing neuromodulation devices, including deep brain stimulation. The descriptive statistics will be summarized for baseline characteristics and related measurements using means (standard deviations) and/or median (interquartile ranges) for continuous data and frequencies and proportions for discrete data. The primary endpoint is the number of T1D patients off insulin therapy achieving fasting BG 70-125 mg/dL and beta-hydroxybutyrate <0.6 mM, so the proportion and corresponding 90% confidence interval are to be reported. Similarly, the secondary endpoints are to be reported with the proportions and confidence intervals. The longitudinal trends are examined through the regression analyses and linear mixed methods. If necessary, statistical tests are used, such as t-tests and Wilcoxon tests for continuous data, and binomial tests and chi-square tests for discrete data to compare means and proportions. The two-sided 5% type I error are applied for the significance levels. The software of SAS (V. 9.4) and R (v. 4.1.0) are used for descriptive and inferential statistics.

### Quality Assurance

Safety oversight is under the direction of a Data and Safety Monitoring Board (DSMB) composed of individuals with the appropriate expertise, which may include neurosurgery, endocrinology, and biostatistics. Members of the DSMB is independent from the study conduct and free of conflict of interest, or measures should be in place to minimize perceived conflict of interest. The DSMB is to meet after the first 2 participants are enrolled and have completed through the EOS visit, after 5 participants have completed through the EOS visit and at the end of study. At the time of dose escalation, a written report is to be submitted to the DSMB Chair (or qualified alternate) describing the dose levels, adverse events, safety reports and any DLTs observed. The DSMB Chair reviews the report and provides a written authorization to proceed, or to request more information within ˜2 business days. Approval for the dose escalation is obtained prior to implementation. The DMSB operates under the rules of an approved charter that is written and reviewed at the organizational meeting of the DSMB. At this time, each data element that the DSMB needs to assess is to be clearly defined. The DSMB provides its input to the Sponsor-Investigator and report to the IRBs as applicable.

### M. Dissemination of Results and Publication Policy

This study complies with the Clinical Trials Registration and Results Information Submission rule. As such, this trial is registered at ClinicalTrials.gov, and results information from this trial is to be submitted to ClinicalTrials.gov. In addition, every attempt is to be made to publish results in peer-reviewed journals.

### N. Ethics/IRB

The participating facilities are responsible for management of the data and conduct of the study. They are committed to ensuring that the trial is conducted, and data are generated, documented, and reported in compliance with the protocol and the applicable regulatory requirements.

The Sponsor-Investigator selects only qualified investigators to participate in the research study and provides investigators with a study protocol and the information they need to properly conduct the study. The sponsor also assures that monitoring is conducted according to the monitoring plan. IRB approval of the study and FDA approval of the IND application must be obtained prior to study start and this documentation is to be on file. The Sponsor-Investigator promptly notifies both the IRB and the FDA of any significant new information about this investigation. The study is conducted in accordance with legal and regulatory requirements per the FDA and ICH GCP guidelines. The only circumstance in which an amendment may be initiated prior to local IRB/IEC approval is where the change is necessary to eliminate apparent immediate hazards to the subjects. In that event, the investigator must notify the local IRB/IEC and trial facilities in writing immediately after the implementation.

### O. Informed Consent

In obtaining and documenting informed consent, the investigator must comply with applicable regulatory requirements (e.g., 45 CFR Part 46, 21 CFR Part 50, 21 CFR Part 56) and should adhere to ICH GCP. Prior to the beginning of the trial, the investigator should have the IRB's written approval for the protocol and the written informed consent form(s) and any other written information to be provided to the participants. Informed consent is a process that is initiated prior to the individual's agreeing to participate in the study and continues throughout the individual's study participation. Consent forms are Institutional Review Board (IRB)-approved and the participant are asked to read and review the document. The investigator needs to explain the research study to the participant and answer any questions that may arise. A verbal explanation is provided in terms suited to the participant's comprehension of the purposes, procedures, and potential risks of the study and of their rights as research participants. Participants have the opportunity to carefully review the written consent form and ask questions prior to signing. The participants should have the opportunity to discuss the study with their family or surrogates or think about it prior to agreeing to participate. The participant signs the informed consent document prior to any procedures being done specifically for the study. Participants must be informed that participation is voluntary and that they may withdraw from the study at any time, without prejudice. A copy of the informed consent document is given to the participants for their records. The informed consent process is conducted and documented in the source document (including the date), and the form signed, before the participant undergoes any study-specific procedures. The rights and welfare of the participants are protected by emphasizing to them that the quality of their medical care is not adversely affected if they decline to participate in this study.

### P. Confidentiality

Each subject participating in this study is assigned a unique identifier. The Investigator agrees to comply with all applicable federal, state, and local laws and regulations relating to the privacy of subjects' health information, including, but not limited to, the Standards for Individually Identifiable Health Information, 45CFR Parts160 and 164 (the Health Insurance Portability and Accountability Act of 1996 privacy regulation). The Investigator shall ensure that all data shall be secured against unauthorized access. Privacy and confidentiality of information about each subject shall be preserved in study reports and in any publication.

### Q. Future Use of Stored Specimens and Data

Data collected for this study is analyzed and stored at the Barrow Neurological Institute (BNI). After the study is completed, the de-identified, archived data is to be transmitted to and stored at the Data Repository, for use by other researchers including those outside of the study. Permission to transmit data to the Data Repository is included in the informed consent.

With the participant's approval and as approved by local Institutional Review Boards (IRBs), de-identified biological samples is stored at BNI with the same goal as the sharing of data with the Data Repository. These samples could be used to research the causes of T1D, its complications, and other conditions for which individuals with T1D are at increased risk, and to improve treatment. The Data Repository will also be provided with a code-link that will allow linking the biological specimens with the phenotypic data from each participant, maintaining the blinding of the identity of the participant.

During the conduct of the study, an individual participant can choose to withdraw consent to have biological specimens stored for future research. However, withdrawal of consent with regard to biosample storage may not be possible after the study is completed.

### Expected Outcome

Outcome of this proof-of-concept study may demonstrate that in T1D patients, brain-directed therapies can restore the defended level of glycemia to normal without insulin. Such outcome would open entirely new therapeutic approaches for these patients. In addition to central leptin administration, other therapeutic strategies targeting glucoregulatory neurocircuits with the potential to restore normoglycemia will become more tractable, feasible, and garner increased research attention. The present disclosure represents the direct translation of a basic research finding replicated in multiple independent labs into a clinical application in human patients with T1D. The current availability of FDA-approved implantable pumps for intrathecal drug delivery, indicated and used widely for intractable pain and spasticity, means that positive results from this feasibility study would be immediately poised for larger, chronic outpatient studies aimed at therapeutic translation in T1D patients.

### Example 5. Effectuate Brain Update Through Systemic Delivery

The present disclosure also relates to delivering a therapeutically effective amount of the leptin active to the brain through a systemic delivery. The systemic delivery includes an intravenous injection, an intramuscular injection, a subcutaneous injection, an oral delivery, a buccal delivery, or a transdermal delivery. In order to effectuate brain uptake and/or accumulation, and to overcome BBB, one or more excipient capable of enhancing brain uptake are used, including osmotic agents, osmogenic agents, sugars, inorganic salts. Specifically, the excipient may include mannitol, glycerol, lactulose, sorbitol, polyethylene glycol, glutamic acid, glycine, polysorbate 20, sucrose, sodium chloride, potassium chloride, magnesium, a magnesium salt, or any combination thereof.

Additionally, a modified leptin compound is a conjugate of the leptin compound and a BBB penetrating moiety. A BBB penetrating moiety comprises at least one fragment selected from the group consisting of a transferrin receptor, an insulin receptor, an insulin-like growth factor receptor, a low density lipoprotein receptor, a low density lipoprotein receptor-related protein 8, a low density lipoprotein receptor-related protein 1, a heparin-binding epidermal growth factor-like growth factor, an antibody thereof, or any combination thereof.

The systemic delivery may be in the form of intravenous administration, intramuscular administration, intrathecal administration, intraperitoneal administration, intrapulmonary administration, transdermal administration, subcutaneous administration, or oral administration. By selecting the most favorable excipients or carriers for brain uptake and/or accumulation, the present invention would allow patients to receive the therapeutically effective amount of the leptin active without the burden of surgical delivery.

### Example 5. Discussions

Insulin therapy, either through peripheral self-administered injection or subcutaneous insulin pump with frequent blood glucose monitoring either with glucose test strips or a continuous glucose monitoring device. The present disclosure avoids glucose monitoring and insulin therapy. It allows treatment of hyperglycemia in Type 1 Diabetes without insulin, which inherently eliminates the risk of hypoglycemia and weight gain and excludes the need for frequent blood glucose monitoring. This is the first treatment for Type 1 Diabetes that targets the brain/central nervous system (CNS). Current insulin therapy requires very active participation by the patient to monitor their blood glucose level and administer insulin in a reactive manner, with insulin dose measured to be appropriate for not only the blood glucose level, but also the anticipated nutritional state and activity level. With CNS leptin administration, blood glucose levels normalize and stay within the normal range so long as leptin is delivered at or above the threshold dose, with no risk of hypoglycemia at doses above the threshold dose. The normalizing effect on blood glucose is stable as long as leptin continues to be administered at or above the threshold dose. Other advantages include more stable blood glucose control, no risk of hypoglycemia, no weight gain, reduction or elimination of frequent blood glucose monitoring, and the potential for reduction of long-term costs associated with Type 1 Diabetes.

The present therapy requires a minimally invasive procedure to 1) place the intrathecal catheter via a lumbar puncture (i.e., “a spinal tap”) and 2) to place the subcutaneous reservoir or pump that will deliver the leptin via the catheter directly to the CNS. The present therapy is open to use both new and existing delivery tools. New delivery tools are designed for intrathecal delivery of leptin, such as in the form of a catheter, a reservoir, or a pump. Additionally, the invention may call to repurpose the existing products that are currently used for intrathecal delivery of other pharmaceutically active ingredients, such as pain medications used in people with chronic pain. Such devices include products from Medtronic and/or Flowonix.

The present study would help T1D patients tremendously if the brain-directed therapies can restore the defended level of glycemia to normal without insulin. Such outcome would open entirely new therapeutic approaches for these patients. In addition to central leptin administration, other therapeutic strategies targeting glucoregulatory neurocircuits with the potential to restore normoglycemia will become more tractable, feasible, and garner increased research attention. The present disclosure represents the direct translation of a basic research finding replicated in multiple independent labs into a clinical application in human patients with T1D. The current availability of FDA-approved implantable pumps for intrathecal drug delivery, indicated and used widely for intractable pain and spasticity, means that positive results from this feasibility study is immediately poised for larger, chronic outpatient studies aimed at therapeutic translation in T1D patients.

We claim:

1. A method of treating a glucose metabolism disorder in a subject in need thereof, comprising:
administering to the subject a therapeutically effective amount of a leptin active, wherein the administering comprises delivering the leptin active to the central nervous system.

2. The method of claim 1, wherein the glucose metabolism disorder is selected from hyperglycemia, Type I diabetes, Type II diabetes, and any combination thereof.

3. The method of claim 1, wherein the leptin active is selected from the group consisting of a leptin compound, a modified leptin compound, a pharmaceutically acceptable salt thereof, a crystal form thereof, an enantiomer thereof, and any combination thereof, wherein the leptin compound is selected from a leptin, a leptin isomer, a leptin derivative, a leptin analog, a leptin mimetic, a leptin agonist, an anti-leptin receptor antibody, or any combination thereof, and the modified leptin compound is a conjugate between the leptin compound and a Blood-Brain-Barrier (BBB) penetrating moiety.

4. The method of claim 3, wherein the BBB penetrating moiety comprises:
at least one fragment selected from the group consisting of a transferrin receptor, an insulin receptor, an insulin-like growth factor receptor, a low-density lipoprotein receptor, a low-density lipoprotein receptor-related protein 8, a low density lipoprotein receptor-related protein 1, a heparin-binding epidermal growth factor-like growth factor, an antibody thereof, or any combination thereof.

5. The method of claim 1, wherein the delivery is to a region of the brain or the spinal cord.

6. The method of claim 5, wherein the region of the brain includes but is not limited to the arcuate nucleus, the ventromedial nucleus of the hypothalamus, or the nucleus of the solitary tract of the hindbrain.

7. The method of claim 1, wherein the therapeutically effective amount of the leptin active is about 5.0 mcg/kg/day to about 10.0 mcg/kg/day.

8. The method of claim 1, wherein the delivery is once daily, once every two days, once every three days, once every four days, once every five days, once every six days, weekly or monthly.

9. The method of claim 1, wherein the delivery is selected from once daily over a period of time in a fixed dose, once daily over a period of time in ascending doses, once daily over a period of time in descending doses, and once daily over a period of time in fluctuating doses.

10. The method of claim 1, wherein the delivery to the central nervous system is achieved by systemic delivery of the leptin active to the subject.

11. The method of claim 10, wherein the systemic delivery is achieved by a route of administration selected from intravenous, intramuscular, subcutaneous, oral, buccal and transdermal.

12. The method of claim 1, wherein the region of the spinal cord is the lumbar region or the cervical region.

13. The method of claim 12, wherein the delivery is achieved via a catheter, a reservoir, or a pump.

14. The method of claim 12, wherein the delivery is through an intrathecal catheter.

15. The method of claim 1, wherein the administering continues for a period of at least 5 days, at least 6 days, least 7 days, at least 8 days, least 9 days, at least 10 days, at least 11 days, at least 12 days, or longer.

16. The method of claim 1, wherein the subject has or is suspected of having Type I diabetes or autoimmune Type I diabetes.

17. The method of claim 1, wherein the subject has or is suspected of having hypoglycemia associated with autonomic failure or obesity.

18. A pharmaceutical composition, comprising
(i) a leptin active; and
(ii) at least one excipient capable of effectuating and/or enhancing the brain uptake of the leptin active;
wherein the leptin active is selected from the group consisting of a leptin compound, a modified leptin compound, a pharmaceutically acceptable salt thereof, a crystal form thereof, an enantiomer thereof, and any combination thereof;
wherein the leptin compound is selected from the group consisting of a leptin, a leptin isomer, a leptin derivative, a leptin analog, a leptin mimetic, a leptin agonist, an anti-leptin receptor antibody, or any combination thereof; and
wherein the modified leptin compound comprises a conjugate of the leptin compound with a Blood-Brain-Barrier (BBB) penetrating moiety.

19. The pharmaceutical composition of claim 18, wherein the BBB penetrating moiety comprises at least one fragment selected from the group consisting of a transferrin receptor, an insulin receptor, an insulin-like growth factor receptor, a low-density lipoprotein receptor, a low density lipoprotein receptor-related protein 8, a low-density lipoprotein receptor-related protein 1, a heparin-binding epidermal growth factor-like growth factor, an antibody thereof, or any combination thereof.

20. The pharmaceutical composition of claim 18, wherein the at least one excipient is selected from the group consisting of an osmotic agent, an osmogenic agent, a sugar, an inorganic salt, and any combination thereof.

21. The pharmaceutical composition of claim 18, wherein the at least one excipient comprises mannitol, glycerol, lactulose, sorbitol, polyethylene glycol, glutamic acid, glycine, polysorbate 20, sucrose, sodium chloride, potassium chloride, magnesium, a magnesium salt, or any combination thereof.

22. The pharmaceutical composition of claim 18, further comprising an excipient or a carrier wherein the composition is formulated for intravenous administration, for intramuscular administration, for intrathecal administration, for intraperitoneally administration, for intrapulmonary administration, for transdermal administration, for subcutaneous administration, or for oral administration.

23. The pharmaceutical composition of claim 18, wherein the pharmaceutical composition comprises an injectable, a tablet, a capsule, a patch, an implant, or a depot.

24. A method of treating a glucose metabolism disorder in a subject in need thereof, comprising:
administering to the subject a therapeutically effective amount of the pharmaceutical composition of any one of claim 18, wherein the administering comprises delivering the leptin active to a region of the central nervous system.

25. The method of claim 24, wherein the glucose metabolism disorder is selected from the group consisting of hyperglycemia, Type I diabetes and Type II diabetes.

26. A leptin compound or a modified leptin compound, derived and/or modified from the amino acid sequence as set forth in SEQ ID NO:1.
